(19) United States
(12) Patent Application Publication
(10) Pub. No.: US 2026/0258512 A1
(43) Pub. Date: Sep. 3, 2026

(54) METHOD FOR DIRECT MICROBIAL IDENTIFICATION

(71) Applicant: Quest Diagnostics Investments LLC, Secaucus, NJ (US)

(72) Inventors: Erik P. JOHNSON, Oceanside, CA (US); Nik ISELY, Moraga, CA (US); Jamie L. PLATT, San Juan Capistrano, CA (US); Martin SIAW, Irvine, CA (US); Ron M. KAGEN, Rancho Santa Margarita, CA (US); Dale A. SCHWAB, Carlsbad, CA (US)

(21) Appl. No.: 19/675,409

(22) Filed: May 12, 2026

Related U.S. Application Data
(62) Division of application No. 17/892,819, filed on Aug. 22, 2022, now Pat. No. 12,655,485.
Application No. 17/892,819 is a division of application No. 15/315,877, filed on Jun. 4, 2015, now Pat. No. 11,421,285.
(60) Provisional application No. 62/007,663, filed on Jun. 4, 2014.

(51) Int. Cl. C12Q 1/689; C12Q 1/686; C12Q 1/6869; C12Q 1/6895; G16B 30/00

(52) U.S. Cl. CPC C12Q 1/689; C12Q 1/686; C12Q 1/6869; C12Q 1/6895; G16B 30/00; C12Q 2600/158; C12Q 2600/16

(57) ABSTRACT

Described herein are methods for direct detection of microbial agent(s) in a polymicrobial sample, such as a biological sample from a human, without culturing the microbial agent(s). The direct detection can identify mixtures of bacteria and/or fungi in the sample. Also described are primer sequences and amplification techniques for performing the direct detection methods.

### CROSS-REFERENCE TO RELATED APPLICATIONS

This application is a Divisional of U.S. application Ser. No. 17/892,819, filed Aug. 22, 2022, which is a Divisional of U.S. application Ser. No. 15/315,877, filed Dec. 2, 2016, now U.S. Pat. No. 11,421,285, issued Aug. 23, 2023, which is a 371 National Stage Entry of PCT/US2015/034202, filed Jun. 4, 2015, which claims priority to U.S. Provisional Application No. 62/007,663, filed Jun. 4, 2014.

### SEQUENCE LISTING

The instant application contains a Sequence Listing that has been submitted electronically in XML format and is hereby incorporated by reference in its entirety. Said XML copy, created on Dec. 6, 2022, and is named 17892819_2_1.xml, and is 450,560 bytes in size.

### FIELD OF THE INVENTION

Methods for direct detection of microbial agent(s) in a sample, including a mixture of bacterial and fungal microbial agents, are disclosed. Nucleotide sequences and amplification techniques to identify microbial agent(s) in a sample also are described.

### BACKGROUND OF THE INVENTION

The following description of the background of the invention is provided simply as an aid in understanding the invention and is not admitted to describe or constitute prior art to the invention.

Microbial agents are currently identified by first culturing the agents using media and growth conditions, and then analyzing morphological/biochemical characteristics or DNA sequencing to determine their identity. Culturing isolates the microbial agents so they can be characterized by phenotypic or genotypic methods, and also provides conditions favorable to grow the microbial agents to produce enough material for analysis.

However, culturing has drawbacks. For example, culturing microbial agents is time consuming and not practical in situations where many different agents are presented in a sample. Moreover, recovering microbial agents from culture can be difficult if the culture ions are not optimized, proper growth conditions are unknown, or certain agents are overgrown and mask the presence of slow-growing agents. The masking of some microbial agents can prevent correctly identifying all microbial agents in a sample.

The masking of some microbial agents is especially problematic with a biofilm sample (e.g., from a chronic wound, a catheter site infection, or due to periodontal disease) because multiple microbial agents can comprise the biofilm, but the most pathogenic specie(s) may be present in the lowest abundance. As a result, a patient's microbial infections are often treated with antibiotics that are not effective in treating their particular infection because the particular pathogenic species is unknown.

A technique to quickly identify all microbial agents in a sample would allow for quicker and more accurate identification of the source(s) of a microbial infection.

### SUMMARY OF THE INVENTION

Provided herein are methods for determining the presence or absence of a microbial agent in a sample, comprising (a) contacting a sample containing sample nucleic acids with an amplification reaction mixture, wherein the amplification reaction mixture primers that specifically amplify at least one target sequence of bacterial 16S rDNA, at least one target sequence of fungal ITS rDNA, and at least one target sequence selected each of Mycobacterium rpoB, Staphylococcus rpoB, Streptococcus rpoB, Burkholderia recA, Enterococcus tuf, and Pseudomonas gvrB, the generate amplification reaction mixture containing the sample nucleic acids; (b) subjecting the amplification reaction mixture containing the sample nucleic acids to polymerase chain reaction (PCR) conditions to generate microbial amplicons; (c) producing adapter-tagged amplicons by attaching the microbial amplicons of step (b), if present, to nucleic acid adapters; (d) amplifying the adapter-tagged amplicons, if present, from step (c) to generate adapter-tagged amplicons; and (e) sequencing the adapter-tagged amplicons, if present, from step (c), wherein a microbial agent is determined to be present in the sample if a microbial amplicon is present and the sequence of the non-adapter portion of an adapter tagged microbial amplicon is at least 90% identical to a nucleotide fragment of bacterial 16S rDNA or fungal ITS rDNA. In some embodiments, the method further comprises identifying the species of bacteria and/or fungus in the sample as Mycobacterium, Staphylococcus, Streptococcus, Burkholderia, Enterococcus and/or Pseudomonas gvrB. In some embodiments, the reagent mixture further comprises a DNA polymerase and a plurality of free nucleotides comprising adenine, thymine, cytosine and guanine. In some embodiments, the PCR involves (i) heating the reaction mixture to a first predetermined temperature for a first predetermined time to separate the strands of the double stranded DNA from each other, (ii) cooling the reaction mixture to a second predetermined temperature for a second predetermined time under conditions to allow the first and second primers to hybridize with their complementary sequences on the first and second strands of the target DNA, and to allow Taq polymerase to extend the primers, and (iii) repeating steps (i) and (ii) at least 12 times to amplify microbial nucleic acids, if present, in the sample to produce microbial amplicons.

In some embodiments, BLAST (Basic Local Alignment Search Tool) is performed to make a broad identification based on the universal rDNA sequence followed by a BLAST of the taxon specific genes to provide resolution to species level.

In some embodiments, a post-extraction step is performed on the sample nucleic acids to remove human DNA prior to combining with the amplification reaction mixture.

In some embodiments, the amplification reaction mixture comprises primers comprising any of SEQ ID NOs 1-335. In some embodiments, multiple different target regions are amplified in a multiplexed reaction. In some embodiments, each target sequence amplification is performed in a separate, individual PCR reaction.

In some embodiments, primers that specifically amplify at least one target sequence of bacterial 16S rDNA comprise a sequence selected from among SEQ ID NOs 89-103.

In some embodiments, primers that specifically amplify at least one target sequence of fungal ITS rDNA comprise a sequence selected from among SEQ ID NOs 119-128.

In some embodiments, primers that specifically amplify at least one target sequence of Mycobacterium rpoB comprise a sequence selected from among SEQ ID NOS 139-152.

In some embodiments, primers that specifically amplify at least one target sequence of Streptococcus rpoB comprise a sequence selected from among SEQ ID NOs 181-233.

In some embodiments, primers that specifically amplify at least one target sequence of Staphylococcus rpoB comprise a sequence selected from among SEQ ID NOs 273-298.

In some embodiments, primers that specifically amplify at least one target sequence of Burkholderia recA comprise a sequence selected from among SEQ ID NOs 299-306.

In some embodiments, primers that specifically amplify at least one target sequence of Enterococcus tuf comprise a sequence selected from among SEQ ID NOs 307-312.

In some embodiments, primers that specifically amplify at least one target sequence of Pseudomonas gvrB comprise a sequence selected from among SEQ ID NOs 313-320.

In some embodiments, the primers further comprise a tag sequence selected from the group consisting of SEQ ID NO:3 and SEQ ID NO:4.

In some embodiments, the adapter sequence is selected from the group consisting of SEQ ID NO: 1 and SEQ ID NO:2.

In some embodiments, the adapters are attached via a primer comprising the adaptor sequence. In some embodiments, the primer comprising the adaptor sequence further comprises a multiplex identifier sequence. In some embodiments, the primer comprising the adaptor sequence further comprises a tag sequence specific for the microbial amplicon. In some embodiments, the tag sequence is selected from the group consisting of SEQ ID NO:3 and SEQ ID NO:4.

In some embodiments, the adapters are attached via enzyme ligation.

In some embodiments, the sample nucleic acids are nucleic acids from a human biological sample. In some embodiments, the biological sample is a urine, sputum, vaginal fluid, sperm, blood or synovial fluid sample

Kits are also provided that comprise at least one of the oligonucleotide primers selected from the group consisting of SEQ ID NOs 1-335.

In some embodiments, the primers in a kit as disclosed herein further comprise a multiplex identifier sequence, a tag sequence and/or an adapter sequence. In some embodiments, one primer of a primer pair comprises an MID and both primers in a primer pair comprise adapter sequences. A forward primer and a reverse primer may comprise different adapter sequences. In some embodiments, the adapter sequence is selected from the group consisting of SEQ ID NO: 1 and SEQ ID NO:2. In some embodiments, the primers further comprise a multiplex identifier sequence. In some embodiments, the primers comprises a tag sequence specific for the microbial amplicon. In some embodiments, the tag sequence is selected from the group consisting of SEQ ID NO:3 and SEQ ID NO:4.

### BRIEF DESCRIPTION OF THE FIGURES

FIG. 1 depicts direct detection of a microbial agent using primers comprising an adapter sequence (Adapter A or Adapter P) and a target specific sequence (SS), with or without a multiplex identifier (MID). Microbial amplicons are generated in a first amplification reaction (PCR1) using primers comprising a target specific sequence (SS). Adapters are attached in a second amplification reaction (PCR2) using the primers comprising an adapter sequence (Adapter A or Adapter P) and a target specific sequence (SS), with or without a multiplex identifier (MID).

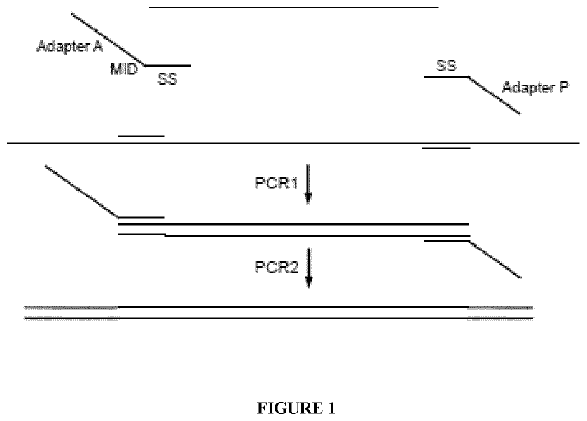

FIG. 2 depicts direct detection of a microbial agent using (i) primers comprising a target specific sequence (SS) and a tag (Tag) and (ii) primers comprising an adapter sequence (Adapter A or Adapter P) and the Tag sequence, with or without a multiplex identifier (MID). Microbial amplicons are generated in a first amplification reaction (PCR1) using primers comprising a target specific sequence (SS) and a tag (Tag). Adapters are attached in a second amplification reaction (PCR2) using the primers comprising an adapter sequence (Adapter A or Adapter P) and the Tag sequence, with or without a multiplex identifier (MID).

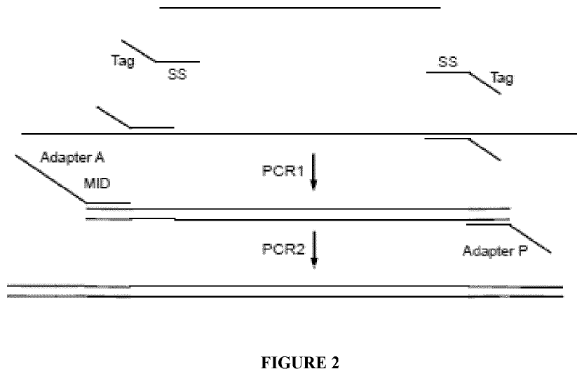

FIG. 3 depicts direct detection of a microbial agent with bi-directional sequencing using (i) primers comprising a target specific sequence (SS) and a tag (Tag) and (ii) primers comprising an adapter sequence (Adapter A or Adapter P) and the Tag sequence, with or without a multiplex identifier (MID). FIG. 3 differs from FIG. 2 in that the adapters are attached in the opposite orientation.

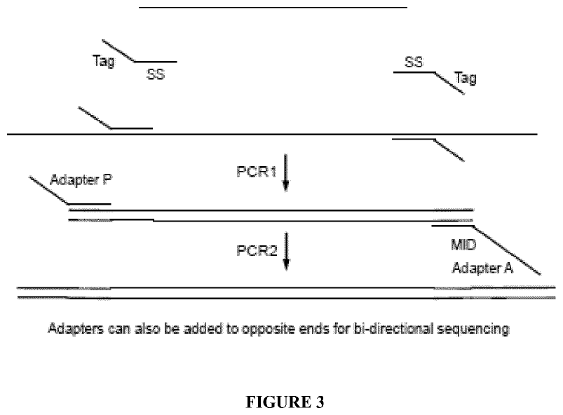

FIG. 4 depicts direct detection of a microbial agent using primers comprising a target specific sequence (SS) in a first amplification reaction (PCR1) and attaching a double stranded adapter sequence (Adapter A or Adapter P), with or without a multiplex identifier (MID), to the microbial amplicon using enzyme ligation (Apollo 324 Adapter Attachment). A second amplification reaction can be performed (PCR1) to further amplify the adapter-tagged amplicon.

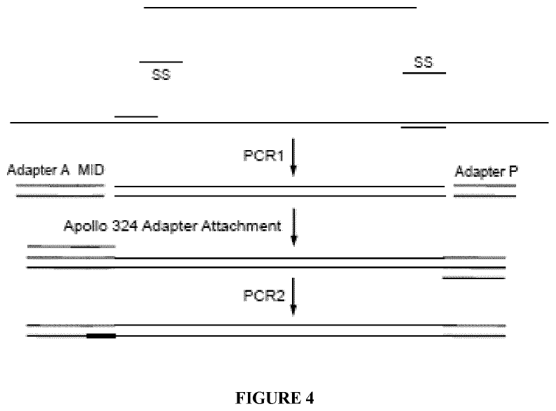

### DETAILED DESCRIPTION OF THE INVENTION

### Definitions

The term “amplify” as used herein with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 Jun. 1; 29 (1 1): E54-E54; Hafner et al., Biotechniques 2001 April;30 (4): 852-6, 858, 860 passim; Zhong et al., Biotechniques 2001 April;30 (4): 852-6, 858, 860.

A “nucleic acid” as used herein refers to a nucleic acid that contains a sequence of a microbial gene, mRNA, cDNA or a portion of such a sequence. A nucleic acid may contain the coding region. A nucleic acid may be genomic DNA, cDNA, single stranded DNA or mRNA. In some embodiments, only a single strand of a sample nucleic acid is amplified and/or sequenced. In some embodiments both strands of double stranded DNA are amplified and sequenced. A nucleic acid may be present in a sample, such as a biological sample, or it may be isolated from the sample.

The term “sense strand” as used herein means the strand of double-stranded DNA (dsDNA) that includes at least a portion of a coding sequence of a functional protein. “Anti-sense strand” means the strand of dsDNA that is the reverse complement of the sense strand.

The terms “complementary” or “complementarity” as used herein with reference to polynucleotides (i.e., a sequence of nucleotides such as an oligonucleotide or a target nucleic acid) refers to the base-pairing rules. The complement of a nucleic acid sequence as used herein refers to nucleotide which, when aligned with the nucleic acid sequence such that the 5′ end of one sequence is paired with the 3′ end of the other, is in “antiparallel association.” For example, the sequence “5′-A-G-T-3” is complementary to the sequence “3′-T-C-A-5.” Certain bases not commonly found in natural nucleic acids may be included in the nucleic acids described herein; these include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA.

The term “substantially complementary” as used herein means that two sequences hybridize under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

The term “hybridize” as used herein refers to a process where two complementary nucleic acid strands anneal to each other under appropriately stringent conditions. Hybridizations are typically and preferably conducted with probe-length nucleic acid molecules, preferably 20-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J. In some embodiments, specific hybridization occurs under stringent hybridization conditions.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

The term “dosage” or “gene dosage” refers to the number of copies of a gene, or portions of a gene, present in a sample.

The term “primer” as used herein means a sequence of nucleic acid, including DNA, which hybridizes to a substantially complementary target sequence and is recognized by DNA polymerase to begin DNA replication. The term primer as used herein includes all forms of primers that may be synthesized, including peptide nucleic acid primers, locked nucleic acid primers, phosphorothioate modified primers, labeled primers, and the like.

The term “forward primer” as used herein means a primer that anneals to the anti-sense strand of dsDNA. A “reverse primer” anneals to the sense-strand of dsDNA.

The term “specific” as used herein in reference to an oligonucleotide primer means that the primer hybridization sequence of the primer has at least 12 bases of sequence identity with a portion of the nucleic acid to be amplified when the oligonucleotide and the nucleic acid are aligned. A primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

The term “flanking” as used herein with regard to primers means that a primer hybridizes to a target nucleic acid adjoining a region of interest sought to be amplified on the target. The skilled artisan will understand that preferred primers are pairs of primers that hybridize 5′ from a region of interest, one on each strand of a target double stranded DNA molecule, such that nucleotides may be added to the 3′ end of the primer by a suitable DNA polymerase. Primers that flank an exon are generally designed not to anneal to the exon sequence but rather to anneal to sequence that adjoins the exon (e.g., intron sequence). However, in some cases, an amplification primer may be designed to anneal to the exon sequence.

“Sequencing depth” or “read depth” as used herein refers to the number of times a sequence has been sequenced (i.e., the depth of sequencing). As an example, read depth can be determined by aligning multiple sequencing run results and counting the start position of reads in nonoverlapping windows of a certain size (e.g., 100 bp). Copy number variation can be determined based on read depth using methods known in the art. For example, using a method described in Yoon et al., Genome Research 2009 September; 19 (9): 1586-1592; Xie et al., BMC Bioinformatics 2009 Mar. 6; 10:80; or Medvedev et al., Nature Methods 2009 November;6 (11 Suppl): S13-20. Use of this type of method and analysis is referred to as a “read depth approach.”

“Coverage depth” refers to the number of nucleotides from sequencing reads that are mapped to a given position.

The term “isolated” as used herein with respect to a nucleic acid (e.g., RNA, DNA or a mixed polymer) is one which is substantially separated from other cellular components which naturally accompany such nucleic acid. The term embraces a nucleic acid sequence which has been removed from its naturally occurring environment, and includes recombinant or cloned DNA isolates, oligonucleotides, and chemically synthesized analogs or analogs biologically synthesized by heterologous systems.

The term “substantially pure” as used herein means a nucleic acid, represents more than 50% of the nucleic acid in a sample. The nucleic acid sample may exist in solution or as a dry preparation.

The term “coding sequence” as used herein means a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the encoding sequence can be deduced there from.

The term “non-coding sequence” as used herein means a sequence of a nucleic acid or its complement, or a part thereof, which is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc. The term “about” as used herein means in quantitative terms plus or minus 10%.

### METHODS

Described herein are methods for direct detection of one or more microbial agents (i.e., microbial agent(s)) in a sample. Direct detection refers to identifying microbial agent(s) in a sample without culturing the sample. Culturing as used herein refers to any technique in which microbial agents in a sample are sustained and/or expanded in vitro, for example, using media and/or growth conditions. In some embodiments, direct detection refers to identifying a mixture of different microbial agents in a sample, such as a mixture of different bacteria, a mixture of different fungi, and a mixture of bacterium/bacteria and fungus/fungi.

In some embodiments, methods for direct detection include extracting nucleic acid from a sample without separating different types of nucleic acid, such as nucleic acid from different types of microbial agents. In some embodiments, methods for direct detection include identifying microbial agent(s) in a sample after extracting nucleic acid from the sample. In specific embodiments, direct detection includes identifying microbial agent(s) in a mammalian biological sample, such as a human biological sample, after extracting nucleic acid from the sample. In other embodiments, direct detection includes identifying microbial agent(s) in a human biological sample after human nucleic acid has been separated and removed from extracted nucleic acid.

### Microbial Agent

A microbial agent as used herein is any microorganism. In some embodiments, the microbial agent is a bacterium. In other embodiments, the microbial agent is a fungus. In some embodiments, the microbial agent is a species selected from the group consisting of Mycobacterium, Streptococcus, Staphylococcus, Burkholderia, Enterococcus, and Pseufomonas.

A target sequence as described herein may represent one or more individual exon(s) or portion(s) of exon(s) of a microbial gene or one or more portions of a microbial mRNA. A target sequence also may include the promoter region and/or one or more introns of a microbial agent gene.

In some embodiments the target sequence represents the entire gene or the entire coding region. In some embodiments, the target sequence represents the entire coding region and at least one intron or a portion thereof and an adjacent region located immediately upstream (in the 5′ direction) of the coding sequence. The adjacent, upstream region may consist of from about 100 nucleotides up to about 500, 750, 1000, 1100, or 1200 nucleotides of the sequence located immediately upstream of the coding sequence. In some embodiments, the adjacent, upstream region comprises all or a portion of the promoter sequence.

### Sample

A sample as used herein contains nucleic acid of microbial agent(s) in, or isolated from, any source. In some embodiments, the sample is a biological sample from a mammal. In specific embodiments, the mammal is a human.

In some embodiments, the biological sample is a body fluid or a tissue sample. In some embodiments the biological sample consists or comprises blood, plasma, sera, urine, feces, epidermal sample, vaginal sample, skin sample, cheek swab, sperm, amniotic fluid, cultured cells, bone marrow sample and/or chorionic villi, cultured cells, and combinations thereof.

In some embodiments, the biological sample is a fixed or frozen tissue. In some embodiments, the biological sample is whole blood of about 0.5 to 5 ml collected with EDTA, ACD or heparin as anti-coagulant. In some embodiments, the biological sample is amniotic fluid of 10-15 ml, cultured cells which are 80-100% confluent in two T-25 flasks, or 25 mg of chorionic villi.

In some embodiments, the sample contains one or more microbial agents. In some embodiments, the sample contains multiple microbial agents. In some embodiments, the sample contains a mixture of bacteria. In other embodiments, the sample contains a mixture of fungi. In other embodiments, the sample contains a mixture of bacterium/bacteria and fungus/fungi.

Processing methods to release or otherwise make available a nucleic acid for detection are well known in the art and may include steps of nucleic acid manipulation, e.g., preparing a cDNA by reverse transcription of RNA from a biological sample. In some embodiments,) a sample taken from a patient is extracted using the MagNA Pure LC instrument or an equivalent tabletop instrument that performs rapid, cross-contamination-free preparation of nucleic acids and PCR setup. The instrument may utilize magnetic-bead technology and may be equipped with a robotic system and automatically isolates any type of nucleic acid. It further may be capable of processing up to 32 different samples in one batch. The enables consistent isolation of high-quality DNA or RNA.

### Adapter Sequence

An adapter sequence (also referred to as a sequencing adapter) is ligated to the 5′ end of the target specific sequence portion of the primer. This sequencing adapter is a short oligonucleotide of known sequence that can provide a priming site for both amplification and sequencing of the adjoining, unknown nucleic acid. As such, adapters allow binding of a fragment to a flow cell for high throughput, massively parallel sequencing, as described herein. Any adapter sequence may be included in a primer used in the present invention.

In some embodiments, all forward amplicons (i.e., amplicons extended from forward primers that hybridized with antisense strands of a target segment) contain the same adapter sequence. In some embodiments when double stranded sequencing is performed, all forward amplicons contain the same adapter sequence and all reverse amplicons (i.e., amplicons extended from reverse primers that hybridized with sense strands of a target segment) contain an adapter sequence that is different from the adapter sequence of the forward amplicons.

In some embodiments, the “forward” adapter sequence consists of or comprises: CCATCTCATCCCTGCGTGTCTCCGACTCAG (SEQ ID NO:1) or a sequence 90%, 95%, 97%, 98%, or 99% identical to SEQ ID NO:1, and the reverse adapter sequence consists of or comprises CCTCTCTATGGGCAGTCGGTGAT (SEQ ID NO:2) or a sequence 90%, 95%, 97%, 98%, or 99% identical to SEQ ID NO:2. These sequences are provided in Table 1.

Other adapter sequences are known in the art. Some manufacturers recommend specific adapter sequences for use with the particular sequencing technology and machinery that they offer.

In some embodiments, when adapter-ligated and/or indexed primers are employed to amplify a target segment, the adapter sequence and/or index sequence gets incorporated into the amplicon (along with the target-specific primer sequence) during amplification. Therefore, the resulting amplicons are sequencing-competent and do not require the traditional library preparation protocol. Moreover, the presence of the index tag permits the differentiation of sequences from multiple sample sources.

In some embodiments, sequencing templates (amplicons) are prepared by emulsion-based clonal amplification of target segments using specialized fusion primers (containing an adapter sequence) and capture beads. A single adapter-bound fragment is attached to the surface of a bead, and an oil emulsion containing necessary amplification reagents is formed around the bead/fragment component. Parallel amplification of millions of beads with millions of single strand fragments produces a sequencer-ready library.

In some embodiments, the amplicons constituting the adapter-tagged (and, optionally, indexed) amplicon library are produced by polymerase chain reaction (PCR). In some embodiments, the amplicon library is generated using a multiplexed PCR approach, such as that disclosed in U.S. Pat. No. 8,092,996, incorporated by reference herein in its entirety.

In other embodiments, each nucleic acid target segment may be amplified with non-adapter-ligated and/or non-indexed primers and a sequencing adapter and/or an index sequence may be subsequently ligated to each of the resulting amplicons.

In some embodiments, sequencing by ligation method using a DNA ligase is applied to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method can utilize Life Technologies' SOLiD™ sequencers.

### Multiplex Identifier

In some cases, amplicons from a single sample source further comprise an identical index sequence (also referred to as an index tag, a “barcode” or a multiplex identifier (MID)). In some cases, indexed amplicons are generated using primers (for example, forward primers and/or reverse primers) containing the index sequence. Such indexed primers may be included during library preparation as a “barcoding” tool to identify specific amplicons as originating from a particular sample source. Indexed amplicons from more than one sample source are quantified individually and then pooled prior to sequencing. As such, the use of index sequences permits multiple samples (i.e., samples from more than one sample source) to be pooled per sequencing run and the sample source subsequently ascertained based on the index sequence. Table 1 provides examples of MID sequences used in the methods described herein.

In some embodiments, amplicons from more than one sample source are pooled prior to high throughput sequencing. “Multiplexing” is the pooling of multiple adapter-tagged and indexed libraries into a single sequencing run. When indexed primer sets are used, this capability can be exploited for comparative studies. In some embodiments, amplicon libraries from up to 48 separate sources are pooled prior to sequencing.

### High Throughput, Massively Parallel Sequencing

High throughput, massively parallel sequencing refers to sequencing methods that can generate multiple sequencing reactions of clonally amplified molecules and of single nucleic acid molecules in parallel. This allows increased throughput and yield of data. These methods are also known in the art as next generation sequencing (NGS) methods. NGS methods include, for example, sequencing-by-synthesis using reversible dye terminators, and sequencing-by-ligation.

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing.

Non-limiting examples of commonly used NGS platforms include Apollo 324™ NGS Library Prep System (IntengenX, Pleasanton, United States), Ion Torrent™ (Life Technologies, Carlsbad, CA), miRNA BeadArray (Illumina, Inc.), Roche 454™ GS FLX™-Titanium (Roche Molecular Diagnostics, Germany), and ABI SOLID™ System (Applied Biosystems, Foster City, CA). Following the production of an adapter tagged and, optionally indexed, amplicon library, the amplicons are sequenced using high throughput, massively parallel sequencing.

### Kit and Primer(S)

The direct detection methods as described herein can be performed using a kit comprising any one or more of the following components: universal primer(s) (e.g., 16S rDNA and ITS rDNA); primer(s), including primer(s) comprising one or more of a target specific sequence, adapter sequence, MID, and tag; dNTP; and other components for amplifying nucleic acid, such as by PCR (including via high throughput, massively parallel sequencing). In some embodiments, the kit comprises components to extract human nucleic acid from a sample.

In some embodiments, the kit comprises any one or more of SEQ ID NOs: 1-335, as listed in Tables 1-10. The kit can include a primer or primer pair comprising any combination of the sequences listed in Tables 1-10, with or without additional nucleic acid(s). For example, SEQ ID NO:23 is a primer consisting of SEQ ID NO: 1 (Adapter A sequence) and SEQ ID NO:5 (MID1). However, a primer or primer pair as described herein can include SEQ ID NO:1 and SEQ ID NO:5 with additional nucleic acid(s) between the two sequences or flanking one or both sequences. In some embodiments, a primer or primer pair as described herein comprises a spacer between two or more of SEQ ID NOs: 1-335. Spacers are known in the art.

TABLE 1
Adapter, Tag, and Multiplex Identifier Sequences

| | | |
|---|---|---|
| SEQ ID | | |
| NO: | Name | Sequence |

Adapter Sequences

| | | |
|---|---|---|
| 1 | Adapter A | CCATCTCATCCCTGCGTGTCTCCGACTCAG |
| 2 | Adapter P | CCTCTCTATGGGCAGTCGGTGAT |

Tag Sequences

| | | |
|---|---|---|
| 3 | Forward Tag | ACACTGACGACATGGTTCTACA |
| 4 | Reverse Tag | TACGGTAGCAGAGACTTGGTCT |

Multiplex Index Sequences

| | | |
|---|---|---|
| 5 | MID1 | ACGAGTGCGT |
| 6 | MID2 | ACGCTCGACA |
| 7 | MID3 | AGACGCACTC |
| 8 | MID4 | AGCACTGTAG |
| 9 | MID5 | ATCAGACACG |
| 10 | MID6 | ATATCGCGAG |
| 11 | MID7 | CGTGTCTCTA |
| 12 | MID8 | CTCGCGTGTC |
| 13 | MID9 | TAGTATCAGC |
| 14 | MID10 | TCTCTATGCG |
| 15 | MID11 | TGATACGTCT |
| 16 | MID12 | TACTGAGCTA |
| 17 | MID13 | CATAGTAGTG |
| 18 | MID14 | CGAGAGATAC |
| 19 | MID15 | ATACGACGTA |
| 20 | MID16 | TCACGTACTA |
| 21 | MID17 | CGTCTAGTAC |
| 22 | MID18 | TCTACGTAGC |

Primers with Adapter and Multiplex Index Sequences

| | | |
|---|---|---|
| 23 | PGMA MID1 | CCATCTCATCCCTGCGTGTCTCCGACTCAG ACGAGTGCGT |
| 24 | PGMA MID2 | CCATCTCATCCCTGCGTGTCTCCGACTCAG ACGCTCGACA |
| 25 | PGMA MID3 | CCATCTCATCCCTGCGTGTCTCCGACTCAG AGACGCACTC |
| 26 | PGMA MID4 | CCATCTCATCCCTGCGTGTCTCCGACTCAG AGCACTGTAG |
| 27 | PGMA MID5 | CCATCTCATCCCTGCGTGTCTCCGACTCAG ATCAGACACG |
| 28 | PGMA MID6 | CCATCTCATCCCTGCGTGTCTCCGACTCAG ATATCGCGAG |
| 29 | PGMA MID7 | CCATCTCATCCCTGCGTGTCTCCGACTCAG CGTGTCTCTA |
| 30 | PGMA MID8 | CCATCTCATCCCTGCGTGTCTCCGACTCAG CTCGCGTGTC |
| 31 | PGMA MID9 | CCATCTCATCCCTGCGTGTCTCCGACTCAG TAGTATCAGC |
| 32 | PGMA MID10 | CCATCTCATCCCTGCGTGTCTCCGACTCAG TCTCTATGCG |
| 33 | PGMA MID11 | CCATCTCATCCCTGCGTGTCTCCGACTCAG TGATACGTCT |
| 34 | PGMA MID12 | CCATCTCATCCCTGCGTGTCTCCGACTCAG TACTGAGCTA |
| 35 | PGMA MID13 | CCATCTCATCCCTGCGTGTCTCCGACTCAG CATAGTAGTG |
| 36 | PGMA MID14 | CCATCTCATCCCTGCGTGTCTCCGACTCAG CGAGAGATAC |
| 37 | PGMA MID15 | CCATCTCATCCCTGCGTGTCTCCGACTCAG ATACGACGTA |
| 38 | PGMA MID16 | CCATCTCATCCCTGCGTGTCTCCGACTCAG TCACGTACTA |
| 39 | PGMA MID1 Comp | ACGCACTCGT CTGAGTCGGAGACACGCAGGGATGAGATGG |
| 40 | PGMA MID2 Comp | TGTCGAGCGT CTGAGTCGGAGACACGCAGGGATGAGATGG |
| 41 | PGMA MID3 Comp | GAGTGCGTCT CTGAGTCGGAGACACGCAGGGATGAGATGG |
| 42 | PGMA MID4 Comp | CTACAGTGCT CTGAGTCGGAGACACGCAGGGATGAGATGG |
| 43 | PGMA MID5 Comp | CGTGTCTGAT CTGAGTCGGAGACACGCAGGGATGAGATGG |
| 44 | PGMA MID6 Comp | CTCGCGATAT CTGAGTCGGAGACACGCAGGGATGAGATGG |
| 45 | PGMA MID7 Comp | TAGAGACACG CTGAGTCGGAGACACGCAGGGATGAGATGG |
| 46 | PGMA MID8 Comp | GACACGCGAG CTGAGTCGGAGACACGCAGGGATGAGATGG |
| 47 | PGMA MID9 Comp | GCTGATACTA CTGAGTCGGAGACACGCAGGGATGAGATGG |
| 48 | PGMA MID10 Comp | CGCATAGAGA CTGAGTCGGAGACACGCAGGGATGAGATGG |
| 49 | PGMA MID11 Comp | AGACGTATCA CTGAGTCGGAGACACGCAGGGATGAGATGG |
| 50 | PGMA MID12 Comp | TAGCTCAGTA CTGAGTCGGAGACACGCAGGGATGAGATGG |
| 51 | PGMA MID13 Comp | CACTACTATG CTGAGTCGGAGACACGCAGGGATGAGATGG |
| 52 | PGMA MID14 Comp | GTATCTCTCG CTGAGTCGGAGACACGCAGGGATGAGATGG |
| 53 | PGMA MID15 Comp | TACGTCGTAT CTGAGTCGGAGACACGCAGGGATGAGATGG |
| 54 | PGMA MID16 Comp | TAGTACGTGA CTGAGTCGGAGACACGCAGGGATGAGATGG |

PCR2 Forward Primers
(Primers with Adapter A, MID, and Forward Tag)

| | | |
|---|---|---|
| 55 | PGMA MID1 FT | CCATCTCATCCCTGCGTGTCTCCGACTCAG ACGAGTGCGT |
| | | ACACTGACGACATGGTTCTACA |
| 56 | PGMA MID2 FT | CCATCTCATCCCTGCGTGTCTCCGACTCAG ACGCTCGACA |
| | | ACACTGACGACATGGTTCTACA |
| 57 | PGMA MID3 FT | CCATCTCATCCCTGCGTGTCTCCGACTCAG AGACGCACTC |
| | | ACACTGACGACATGGTTCTACA |
| 58 | PGMA MID4 FT | CCATCTCATCCCTGCGTGTCTCCGACTCAG AGCACTGTAG |
| | | ACACTGACGACATGGTTCTACA |
| 59 | PGMA MID5 FT | CCATCTCATCCCTGCGTGTCTCCGACTCAG ATCAGACACG |
| | | ACACTGACGACATGGTTCTACA |
| 60 | PGMA MID6 FT | CCATCTCATCCCTGCGTGTCTCCGACTCAG ATATCGCGAG |
| | | ACACTGACGACATGGTTCTACA |
| 61 | PGMA MID7 FT | CCATCTCATCCCTGCGTGTCTCCGACTCAG CGTGTCTCTA |
| | | ACACTGACGACATGGTTCTACA |
| 62 | PGMA MID8 FT | CCATCTCATCCCTGCGTGTCTCCGACTCAG CTCGCGTGTC |
| | | ACACTGACGACATGGTTCTACA |
| 63 | PGMA MID9 FT | CCATCTCATCCCTGCGTGTCTCCGACTCAG TAGTATCAGC |
| | | ACACTGACGACATGGTTCTACA |
| 64 | PGMA MID10 FT | CCATCTCATCCCTGCGTGTCTCCGACTCAG TCTCTATGCG |
| | | ACACTGACGACATGGTTCTACA |
| 65 | PGMA MID11 FT | CCATCTCATCCCTGCGTGTCTCCGACTCAG TGATACGTCT |
| | | ACACTGACGACATGGTTCTACA |
| 66 | PGMA MID12 FT | CCATCTCATCCCTGCGTGTCTCCGACTCAG TACTGAGCTA |
| | | ACACTGACGACATGGTTCTACA |
| 67 | PGMA MID13 FT | CCATCTCATCCCTGCGTGTCTCCGACTCAG CATAGTAGTG |
| | | ACACTGACGACATGGTTCTACA |
| 68 | PGMA MID14 FT | CCATCTCATCCCTGCGTGTCTCCGACTCAG CGAGAGATAC |
| | | ACACTGACGACATGGTTCTACA |
| 69 | PGMA MID15 FT | CCATCTCATCCCTGCGTGTCTCCGACTCAG ATACGACGTA |
| | | ACACTGACGACATGGTTCTACA |
| 70 | PGMA MID16 FT | CCATCTCATCCCTGCGTGTCTCCGACTCAG TCACGTACTA |
| | | ACACTGACGACATGGTTCTACA |

Primer with Adapter P and Reverse Tag

| | | |
|---|---|---|
| 71 | Primer P RT | CCTCTCTATGGGCAGTCGGTGAT |
| | | TACGGTAGCAGAGACTTGGTCT |

PCR2 Reverse Primers
(Primers with Adapter A, MID, and Reverse Tag)

| | | |
|---|---|---|
| 72 | PGMA MID1 RT | CCATCTCATCCCTGCGTGTCTCCGACTCAG ACGAGTGCGT |
| | | TACGGTAGCAGAGACTTGGTCT |
| 73 | PGMA MID2 RT | CCATCTCATCCCTGCGTGTCTCCGACTCAG ACGCTCGACA |
| | | TACGGTAGCAGAGACTTGGTCT |
| 74 | PGMA MID3 RT | CCATCTCATCCCTGCGTGTCTCCGACTCAG AGACGCACTC |
| | | TACGGTAGCAGAGACTTGGTCT |
| 75 | PGMA MID4 RT | CCATCTCATCCCTGCGTGTCTCCGACTCAG AGCACTGTAG |
| | | TACGGTAGCAGAGACTTGGTCT |
| 76 | PGMA MID5 RT | CCATCTCATCCCTGCGTGTCTCCGACTCAG ATCAGACACG |
| | | TACGGTAGCAGAGACTTGGTCT |
| 77 | PGMA MID6 RT | CCATCTCATCCCTGCGTGTCTCCGACTCAG ATATCGCGAG |
| | | TACGGTAGCAGAGACTTGGTCT |
| 78 | PGMA MID7 RT | CCATCTCATCCCTGCGTGTCTCCGACTCAG CGTGTCTCTA |
| | | TACGGTAGCAGAGACTTGGTCT |
| 79 | PGMA MID8 RT | CCATCTCATCCCTGCGTGTCTCCGACTCAG CTCGCGTGTC |
| | | TACGGTAGCAGAGACTTGGTCT |
| 80 | PGMA MID9 RT | CCATCTCATCCCTGCGTGTCTCCGACTCAG TAGTATCAGC |
| | | TACGGTAGCAGAGACTTGGTCT |
| 81 | PGMA MID10 RT | CCATCTCATCCCTGCGTGTCTCCGACTCAG TCTCTATGCG |
| | | TACGGTAGCAGAGACTTGGTCT |
| 82 | PGMA MID11 RT | CCATCTCATCCCTGCGTGTCTCCGACTCAG TGATACGTCT |
| | | TACGGTAGCAGAGACTTGGTCT |
| 83 | PGMA MID12 RT | CCATCTCATCCCTGCGTGTCTCCGACTCAG TACTGAGCTA |
| | | TACGGTAGCAGAGACTTGGTCT |
| 84 | PGMA MID13 RT | CCATCTCATCCCTGCGTGTCTCCGACTCAG CATAGTAGTG |
| | | TACGGTAGCAGAGACTTGGTCT |
| 85 | PGMA MID14 RT | CCATCTCATCCCTGCGTGTCTCCGACTCAG CGAGAGATAC |
| | | TACGGTAGCAGAGACTTGGTCT |
| 86 | PGMA MID15 RT | CCATCTCATCCCTGCGTGTCTCCGACTCAG ATACGACGTA |
| | | TACGGTAGCAGAGACTTGGTCT |
| 87 | PGMA MID16 RT | CCATCTCATCCCTGCGTGTCTCCGACTCAG TCACGTACTA |
| | | TACGGTAGCAGAGACTTGGTCT |

Primer with Adapter P and Forward Tag

| | | |
|---|---|---|
| 88 | Primer P FT | CCTCTCTATGGGCAGTCGGTGAT |
| | | ACACTGACGACATGGTTCTACA |

TABLE 2
16S Sequences

| | | |
|---|---|---|
| SEQ | | |
| ID | | |
| NO: | Name | Sequence |

16S V2 Set 2

| | | | |
|---|---|---|---|
| 89 | 16S V2 F101a | GGCGGACGGGTGAGTAA | |
| 90 | 16S V2 F101b | GGCGAACGGGTGAGTAA | |
| 91 | 16S V2 F101c | GGCGGACGGGTGAGTAA | |
| 92 | 16S V2 F101d | GGCGGATGGGTGAGTAA | Lactobacillus |
| 93 | 16S V2 F101e | GGCAAACGGGTGAGTAA | Megasphaera |
| 94 | 16S V2 F101f | GGCGAACGGGCGAGTAA | Mobiluncus |
| 95 | 16S V2 F101g | GGCGAACGGCTGAGTAA | Atopobium |
| 96 | 16S V2 R356a | CACTGCTGCCTCCCGTAG | |
| 97 | 16S V2 R356b | TACTGCTGCCTCCCGTAG | |

16S V3 Set 2

| | | | |
|---|---|---|---|
| 98 | 16S V3 F323a | GACACGGTCCAGACTCCTAC | |
| 99 | 16S V3 F323b | GACACGGCCCAGACTCCTAC | |
| 100 | 16S V3 F323c | GACACGGTCCAAACTCCTAC | Bacillus |
| 101 | 16S V3 F323d | GACACGGCCCAAACTCCTAC | Lactobacillus |
| 102 | 16S V3 F323e | GATACGGCCCAGACTCCTAC | Myco, Mob, |
| | | | Gard |
| 103 | 16S V3 R531a | ATTACCGCGGCTGCTG | |

PCR1 V2 (Tag, Sequence Specific)

| | | |
|---|---|---|
| 104 | Tag V2 F101a | ACACTGACGACATGGTTCTA |
| | | CAGGCGGACGGGTGAGTAA |
| 105 | Tag V2 F101b | ACACTGACGACATGGTTCTA |
| | | CAGGCGAACGGGTGAGTAA |
| 106 | Tag V2 F101c | ACACTGACGACATGGTTCTA |
| | | CAGGCGCACGGGTGAGTAA |
| 107 | Tag V2 F101d | ACACTGACGACATGGTTCTA |
| | | CAGGCGGATGGGTGAGTAA |
| 108 | Tag V2 F101e | ACACTGACGACATGGTTCTA |
| | | CAGGCAAACGGGTGAGTAA |
| 109 | Tag V2 F101f | ACACTGACGACATGGTTCTA |
| | | CAGGCGAACGGGCGAGTAA |
| 110 | Tag V2 F101g | ACACTGACGACATGGTTCTA |
| | | CAGGCGAACGGCTGAGTAA |
| 111 | Tag V2 R356a | TACGGTAGCAGAGACTTGGT |
| | | CTCACTGCTGCCTCCCGTAG |
| 112 | Tag V2 R356b | TACGGTAGCAGAGACTTGGT |
| | | CTTACTGCTGCCTCCCGTAG |

PCR1 V3 (Tag, Sequence Specific)

| | | |
|---|---|---|
| 113 | Tag V3 F323a | ACACTGACGACATGGTTCTA |
| | | CAGACACGGTCCAGACTCCT |
| | | AC |
| 114 | Tag V3 F323b | ACACTGACGACATGGTTCTA |
| | | CAGACACGGCCCAGACTCCT |
| | | AC |
| 115 | Tag V3 F323c | ACACTGACGACATGGTTCTA |
| | | CAGACACGGTCCAAACTCCT |
| | | AC |
| 116 | Tag V3 F323d | ACACTGACGACATGGTTCTA |
| | | CAGACACGGCCCAAACTCCT |
| | | AC |
| 117 | Tag V3 F323e | ACACTGACGACATGGTTCTA |
| | | CAGATACGGCCCAGACTCCT |
| | | AC |
| 118 | Tag V3 R531a | TACGGTAGCAGAGACTTGGT |
| | | CTATTACCGCGGCTGCTG |

TABLE 3
ITS Sequences

| | | |
|---|---|---|
| SEQ | | |
| ID | | |
| NO: | Name | Sequence |

ITS Sequences F and R Primer Pairs

| | | |
|---|---|---|
| 119 | ITS1Fb | AAACTCGGTCATTTAGAGGAAGTAA |
| 120 | ITSR513 | GATGCCGGAACCAAGAGAT |
| 121 | ITSF329 | AACCTCCCACCCGTGTTTAT |
| 122 | ITSR533 | ATTTCGCTGCGTTCTTCATC |
| 123 | ITS1Fb | AAACTCGGTCATTTAGAGGAAGTAA |
| 124 | ITS2b | GCTGCGTTCTTCATCGATG |
| 125 | ITSF569 | ATCGAGTCTTTGAACGCACA |
| 126 | ITSR820 | CCTACCTGATCCGAGGTCAA |
| 127 | ITSF570 | TCGAGTCTTTGAACGCACAT |
| 128 | ITSR828 | CGGGTATCCCTACCTGATCC |

ITS Reading Set (Adapter A, MID or Adapter P)

| | | |
|---|---|---|
| 129 | ITSPGM1FbA | CCATCTCATCCCTGCGTGTCTCCGACTCAG |
| | MID1 | ACGAGTGCGTAAACTCGGTCATTTAGAGGA |
| | | AGTAA |
| 130 | ITSPGMR513P | CCTCTCTATGGGCAGTCGGTGAT |
| | | GATGCCGGAACCAAGAGAT |
| 131 | TTSPGMF329A | CCATCTCATCCCTGCGTGTCTCCGACTCAG |
| | MID1 | ACGAGTGCGTAACCTCCCACCCGTGTTTAT |
| 132 | ITSPGMR533P | CCTCTCTATGGGCAGTCGGTGAT |
| | | ATTTCGCTGCGTTCTTCATC |
| 133 | ITSPGM1FbA | CCATCTCATCCCTGCGTGTCTCCGACTCAG |
| | MID1 | ACGAGTGCGTAAACTCGGTCATTTAGAGGA |
| | | AGTAA |
| 134 | ITSPGM2bP | CCTCTCTATGGGCAGTCGGTGAT |
| | | GCTGCGTTCTTCATCGATG |
| 135 | ITSPGMF569A | CCATCTCATCCCTGCGTGTCTCCGACTCAG |
| | MID1 | ACGAGTGCGTATCGAGTCTTTGAACGCACA |
| 136 | ITSPGMR820P | CCTCTCTATGGGCAGTCGGTGAT |
| | | CCTACCTGATCCGAGGTCAA |
| 137 | ITSPGMF570A | CCATCTCATCCCTGCGTGTCTCCGACTCAG |
| | MID1 | ACGAGTGCGTTCGAGTCTTTGAACGCACAT |
| 138 | ITSPGMR828P | CCTCTCTATGGGCAGTCGGTGAT |
| | | CGGGTATCCCTACCTGATCC |

PCR1 (Tag, Sequence Specific)

| | | |
|---|---|---|
| 335 | Tag ITS1Fb | ACACTGACGACATGGTTCTACA |
| | | AAACTCGGTCATTTAGAGGAAGTAA |
| 336 | Tag ITS2b | TACGGTAGCAGAGACTTGGTCT |
| | | GCTGCGTTCTTCATCGATG |
| 337 | Tag ITSF569 | ACACTGACGACATGGTTCTACA |
| | | ATCGAGTCTTTGAACGCACA |
| 338 | Tag ITSR820 | TACGGTAGCAGAGACTTGGTCT |
| | | CCTACCTGATCCGAGGTCAA |

TABLE 4
Mycobacterium Sequences

| | | | |
|---|---|---|---|
| SEQ | | | |
| ID | | | |
| NO: | Name | Sequence | Description |

Mycobacterium Fragment 1

| | | |
|---|---|---|
| 139 | MycoPGMF2649 | GCAAGGTCACCCCGAAG |
| 140 | MycoPGMR2924 | CGATGACGCCCTTGTTG |
| 141 | MycoPGMF2648 | GGCAAGGTCACCCCGAAGG |
| 142 | MycoPGMR2934 | AGGATCTTGCCGATGACG |

Mycobacterium Fragment 2

| | | | |
|---|---|---|---|
| 143 | MycoPGM2F2898 | GACGCCACGGCAACAAG | |
| 144 | MycoPGM2F2899 | ACGCCACGGCAACAAG | |
| 145 | MycoPGM2R3337 | CAAGTGGTGCAGCTTCAGGATG | Corynebacterium |
| 146 | MycoPGM2R3337d | CARGTGGTGCAGCTTCAKGATG | |
| 147 | MycoPGM2R3169 | GGCGCCGTCGAACAC | |
| 148 | MycoPGM2R3169d | GGCRCCGTCGAACAC | |
| 149 | MycoPGM2R3169a | GGCACCGTCGAACAC | |
| 150 | MycoPGM2R3169b | GGCGCCGTCGAACAC | |

Mycobacterium Fragment 3

| | | |
|---|---|---|
| 151 | MycoPGM2F3148 | CACCCCGGTGTTCGAC |
| 152 | MycoPGM2R3391 | CTGGGTGATCATCGAGTACG |

Fragment 1 Forward Reading Set (Adapter A or Adapter P)

| | | |
|---|---|---|
| 153 | MycoPGMF2649A | CCATCTCATCCCTGCGTGTCTCCGACTCAG |
| | | GCAAGGTCACCCCGAAG |
| 154 | MycoPGMR2924P | CCTCTCTATGGGCAGTCGGTGAT |
| | | CGATGACGCCCTTGTTG |
| 155 | MycoPGMF2648A | CCATCTCATCCCTGCGTGTCTCCGACTCAG |
| | | GGCAAGGTCACCCCGAAGG |
| 156 | MycoPGMR2934P | CCTCTCTATGGGCAGTCGGTGAT |
| | | AGGATCTTGCCGATGACG |
| 157 | MycoPGMF2649A | CCATCTCATCCCTGCGTGTCTCCGACTCAG |
| | MID1 | ACGAGTGCGT GCAAGGTCACCCCGAAG |

Fragment 1 Reverse Reading Set (Adapter P or Adapter A)

| | | |
|---|---|---|
| 158 | MycoPGMF2649P | CCTCTCTATGGGCAGTCGGTGAT |
| | | GCAAGGTCACCCCGAAG |
| 159 | MycoPGMR2924A | CCATCTCATCCCTGCGTGTCTCCGACTCAG |
| | | CGATGACGCCCTTGTTG |
| 160 | MycoPGMF2648P | CCTCTCTATGGGCAGTCGGTGAT |
| | | GGCAAGGTCACCCCGAAGG |
| 161 | MycoPGMR2934A | CCATCTCATCCCTGCGTGTCTCCGACTCAG |
| | | AGGATCTTGCCGATGACG |

Fragment 2 Forward Reading Set (Adapter A or Adapter P)

| | | |
|---|---|---|
| 162 | MycoPGM2F2898A | CCATCTCATCCCTGCGTGTCTCCGACTCAG |
| | | GACGCCACGGCAACAAG |
| 163 | MycoPGM2F2899A | CCATCTCATCCCTGCGTGTCTCCGACTCAG |
| | | ACGCCACGGCAACAAG |
| 164 | MycoPGM2R3337P | CCTCTCTATGGGCAGTCGGTGAT |
| | | CAAGTGGTGCAGCTTCAGGATG |
| 165 | MycoPGM2R3337dP | CCTCTCTATGGGCAGTCGGTGAT |
| | | CARGTGGTGCAGCTTCAKGATG |
| 166 | MycoPGM2R3169P | CCTCTCTATGGGCAGTCGGTGAT |
| | | GGCGCCGTCGAACAC |
| 167 | MycoPGM2R3169dP | CCTCTCTATGGGCAGTCGGTGAT |
| | | GGCRCCGTCGAACAC |
| 168 | MycoPGM2F2898A | CCATCTCATCCCTGCGTGTCTCCGACTCAG |
| | MID1 | ACGAGTGCGT GACGCCACGGCAACAAG |
| 169 | MycoPGM2F2899A | CCATCTCATCCCTGCGTGTCTCCGACTCAG |
| | MID1 | ACGAGTGCGT ACGCCACGGCAACAAG |

Fragment 2 Reverse Reading Set (Adapter P or Adapter A)

| | | |
|---|---|---|
| 170 | MycoPGM2F2898P | CCTCTCTATGGGCAGTCGGTGAT |
| | | GACGCCACGGCAACAAG |
| 171 | MycoPGM2F2899P | CCTCTCTATGGGCAGTCGGTGAT |
| | | ACGCCACGGCAACAAG |
| 172 | MycoPGM2R3337A | CCATCTCATCCCTGCGTGTCTCCGACTCAG |
| | | CAAGTGGTGCAGCTTCAGGATG |
| 173 | MycoPGM2R3337dA | CCATCTCATCCCTGCGTGTCTCCGACTCAG |
| | | CARGTGGTGCAGCTTCAKGATG |
| 174 | MycoPGM2R3169A | CCATCTCATCCCTGCGTGTCTCCGACTCAG |
| | | GGCGCCGTCGAACAC |
| 175 | MycoPGM2R3169dA | CCATCTCATCCCTGCGTGTCTCCGACTCAG |
| | | GGCRCCGTCGAACAC |

Fragment 3 Forward Reading Set (Adapter A or Adapter P)

| | | |
|---|---|---|
| 176 | MycoPGM2F3148A | CCATCTCATCCCTGCGTGTCTCCGACTCAG |
| | | CACCCCGGTGTTCGAC |
| 177 | MycoPGM2R3391P | CCTCTCTATGGGCAGTCGGTGAT |
| | | CTGGGTGATCATCGAGTACG |
| 178 | MycoPGM2F3148A | CCATCTCATCCCTGCGTGTCTCCGACTCAG |
| | MID1 | ACGAGTGCGT CACCCCGGTGTTCGAC |

Fragment 3 Reverse Reading Set (Adapter P or Adapter A)

| | | |
|---|---|---|
| 179 | MycoPGM2F3148P | CCTCTCTATGGGCAGTCGGTGAT |
| | | CACCCCGGTGTTCGAC |
| 180 | MycoPGM2R3391A | CCATCTCATCCCTGCGTGTCTCCGACTCAG |
| | | CTGGGTGATCATCGAGTACG |

TABLE 5
Streptococcus Sequences

| | | | |
|---|---|---|---|
| SEQ | | | |
| ID | | | |
| NO: | Name | Sequence | Description |

Streptococcus Fragment 1

| | | | |
|---|---|---|---|
| 181 | StrepF1475a | CCTTGGGACCTGGTGGTT | saliv therm mitisB |
| | | | oralis suis |
| | | | anginosus |
| 182 | StrepF1475b | CCTTAGGACCTGGTGGTT | pyog dysgalac |
| | | | canis equi agalac |
| | | | pneumo sang |
| 183 | StrepF1475c | GCTTTAGGTCCTGGTGGTT | mutans |
| 184 | StrepF1475d | CCTTGGGGCCTGGTGGTT | mitisB |
| 185 | StrepF1475e | CCTTAGGGCCTGGTGGTT | Parasanguinis |
| 186 | StrepR1720a | CTTCTTCGTCGGCAGTCAAC | saliv therm pyog |
| | | | canis |
| 187 | StrepR1720b | CTTCTTCATCAGCAGTCAACC | pyog2 agalac |
| | | | dysgalac |
| 188 | StrepR1720c | CTTCTTCATCAGCAGTTAGC | equi |
| 189 | StrepR1720d | CTTCTTCATCAGCAGTAAGC | mutans |
| 190 | StrepR1720e | CTTCTTCATCAGCTGTCAAC | pneumo |
| 191 | StrepR1720f | CTTCTTCATCGGCTGTCAAC | mitis oralis paras |
| | | | suis |
| 192 | StrepR1720g | CTTCCTCGTCAGCGGTCAAC | sang |
| 193 | StrepR1720h | CTTCTTCGTCCGCTGTCAGC | anginosus |
| 194 | StrepR1720i | CTTCTTCATCCGCTGTTAGC | intermedius |

Streptococcus Fragment 2

| | | | |
|---|---|---|---|
| 195 | StrepF1875a | TGCGACAGCATGTATTCCTT | |
| 196 | StrepF1875b | CGCAACAGCATGTATTCCTT | agalac |
| 197 | StrepF1875c | TGCAACGGCATGTATTCCTT | pyogenes dysgalac |
| | | | canis |
| 198 | StrepF1875d | GGCAACGGCATGTATTCCTT | intermedius |
| 199 | StrepR2148a | TGAGTTTGAACGACGGAATTT | saliv therm pyog |
| | | | dysgalac pneumo |
| | | | mitisB paras |
| 200 | StrepR2148b | TGAGTTGGAGCGACGGAATTT | canis |
| 201 | StrepR2148c | AGAGTTTGAACGGCGGAATTT | equi anginosus |
| 202 | StrepR2148d | AGAGTTAGAACGACGGAATTT | mutans |
| 203 | StrepR2148e | TGAGTTTGAACGGCGGAATTT | agalactie |
| 204 | StrepR2148f | TGAGTTAGAACGACGGAATTT | mitis oralis |
| 205 | StrepR2148g | TGAGTTAGAACGGCGGAATTT | sang intermedius |

Streptococcus Fragment 3

| | | | |
|---|---|---|---|
| 206 | StrepF2885a | TGAACATCGGTCAGGTTATGG | salivarus suis |
| 207 | StrepF2885b | TGAACATTGGTCAGGTTATGG | thermo dysgalac |
| | | | sanguin |
| 208 | StrepF2885c | TGAATATTGGTCAGGTTATGG | pyogenes |
| 209 | StrepF2885d | TGAATATCGGTCAGGTTATGG | pneumo mitis |
| | | | oralis paras |
| 210 | StrepF2885e | TGAACATCGGACAAGTTATGG | canis |
| 211 | StrepF2885f | TGAACATTGGACAGGTTATGG | equi |
| 212 | StrepF2885g | TGAACATTGGGCAAGTTATGG | mutans |
| 213 | StrepF2885h | TGAATATCGGACAAGTTATGG | agalac intermedius |
| 214 | StrepF2885i | TGAATATTGGTCAAGTTATGG | anginosus |
| 215 | StrepR3134a | TGAAGTTTATCATCAACCATGTG | salivarus thermo |
| | | | pyog dysgal canis |
| | | | suis |
| 216 | StrepR3134b | TGCAATTTATCATCAACCATGTG | mutans mitis oralis |
| 217 | StrepR3134c | TGCAACTTATCATCAACCATGTG | agalac |
| 218 | StrepR3134d | TGAAGCTTATCATCTACCATGTG | intermedius |
| 219 | StrepR3134e | TGGAGTTTATCATCTACCATGTG | sang |
| 220 | StrepR3134f | TGAAGCTTATCATCAACCATGTG | equi |
| 221 | StrepR3134g | TGCAATTTATCGTCAACCATGTG | pneumo |
| 222 | StrepR3134h | TGGAGCTTATCATCAACCATGTG | anginosus |

Streptococcus Fragment 4

| | | | |
|---|---|---|---|
| 223 | StrepF3106a | CTTCACCACATGGTTGATGATAA | saliv thermo pyog |
| | | | mutans paras suis |
| | | | equi |
| 224 | StrepF3106b | CTCCACCACATGGTTGATGATAA | dysgalac canis |
| | | | mitis oralis |
| 225 | StrepF3106c | CTCCACCACATGGTTGACGATAA | pneumo |
| 226 | StrepF3106d | CTCCACCACATGGTAGATGATAA | sang |
| 227 | StrepF3106e | CTTCACCACATGGTAGATGATAA | intermed |
| 228 | StrepR3366a | TTCTGGTACACCTGGTTTTGG | saliv thermo pyog |
| | | | dysgalac paras |
| 229 | StrepR3366b | TTCTGGCACACCTGGTTTTGG | canis sang |
| 230 | StrepR3366c | TTCTGGAACACCTGGTTTTGG | agalac pneumo |
| | | | mitis oralis suis |
| | | | anginosus |
| 231 | StrepR3366d | TTCTGGGACACCTGGTTTTGG | intermed |
| 232 | StrepR3366e | TTCTGGTACACCAGGCTTTGG | equi |
| 233 | StrepR3366f | TTCTGGTACCCCTGGTTTTGG | mutans |

PGM Fragment 2 Set (Adapter A, MID or Adapter P)

| | | |
|---|---|---|
| 234 | StrepPGMF1875a | CCATCTCATCCCTGCGTGTCTCCGACTCAG |
| | MID1 | ACGAGTGCGT |
| 235 | StrepPGMF1875b | CCATCTCATCCCTGCGTGTCTCCGACTCAG |
| | MID1 | ACGAGTGCGT |
| 236 | StrepPGMF1875c | CCATCTCATCCCTGCGTGTCTCCGACTCAG |
| | MID1 | ACGAGTGCGT |
| 237 | StrepPGMF1875d | CCATCTCATCCCTGCGTGTCTCCGACTCAG |
| | MID1 | ACGAGTGCGT |
| 238 | StrepPGMR2148a | CCTCTCTATGGGCAGTCGGTGAT |
| 239 | StrepPGMR2148b | CCTCTCTATGGGCAGTCGGTGAT |
| 240 | StrepPGMR2148c | CCTCTCTATGGGCAGTCGGTGAT |
| 241 | StrepPGMR2148d | CCTCTCTATGGGCAGTCGGTGAT |
| 242 | StrepPGMR2148e | CCTCTCTATGGGCAGTCGGTGAT |
| 243 | StrepPGMR2148f | CCTCTCTATGGGCAGTCGGTGAT |
| 244 | StrepPGMR2148g | CCTCTCTATGGGCAGTCGGTGAT |

PGM Fragment 3 Set (Adapter A, MID or Adapter P)

| | | |
|---|---|---|
| 245 | StrepPGMF2885a | CCATCTCATCCCTGCGTGTCTCCGACTCAG |
| | MID1 | ACGAGTGCGT |
| 246 | StrepPGMF2885b | CCATCTCATCCCTGCGTGTCTCCGACTCAG |
| | MID1 | ACGAGTGCGT |
| 247 | StrepPGMF2885c | CCATCTCATCCCTGCGTGTCTCCGACTCAG |
| | MID1 | ACGAGTGCGT |
| 248 | StrepPGMF2885d | CCATCTCATCCCTGCGTGTCTCCGACTCAG |
| | MID1 | ACGAGTGCGT |
| 249 | StrepPGMF2885e | CCATCTCATCCCTGCGTGTCTCCGACTCAG |
| | MID1 | ACGAGTGCGT |
| 250 | StrepPGMF2885f | CCATCTCATCCCTGCGTGTCTCCGACTCAG |
| | MID1 | ACGAGTGCGT |
| 251 | StrepPGMF2885g | CCATCTCATCCCTGCGTGTCTCCGACTCAG |
| | MID1 | ACGAGTGCGT |
| 252 | StrepPGMF2885h | CCATCTCATCCCTGCGTGTCTCCGACTCAG |
| | MID1 | ACGAGTGCGT |
| 253 | StrepPGMF2885i | CCATCTCATCCCTGCGTGTCTCCGACTCAG |
| | MID1 | ACGAGTGCGT |
| 254 | StrepPGMR3134a | CCTCTCTATGGGCAGTCGGTGAT |
| 255 | StrepPGMR3134b | CCTCTCTATGGGCAGTCGGTGAT |
| 256 | StrepPGMR3134c | CCTCTCTATGGGCAGTCGGTGAT |
| 257 | StrepPGMR3134d | CCTCTCTATGGGCAGTCGGTGAT |
| 258 | StrepPGMR3134e | CCTCTCTATGGGCAGTCGGTGAT |
| 259 | StrepPGMR3134f | CCTCTCTATGGGCAGTCGGTGAT |
| 260 | StrepPGMR3134g | CCTCTCTATGGGCAGTCGGTGAT |
| 261 | StrepPGMR3134h | CCTCTCTATGGGCAGTCGGTGAT |

PGM Fragment 4 Set (Adapter A, MID or Adapter P)

| | | |
|---|---|---|
| 262 | StrepPGMF3106a | CCATCTCATCCCTGCGTGTCTCCGACTCAG |
| | MID1 | ACGAGTGCGT |
| 263 | StrepPGMF3106b | CCATCTCATCCCTGCGTGTCTCCGACTCAG |
| | MID1 | ACGAGTGCGT |
| 264 | StrepPGMF3106c | CCATCTCATCCCTGCGTGTCTCCGACTCAG |
| | MID1 | ACGAGTGCGT |
| 265 | StrepPGMF3106d | CCATCTCATCCCTGCGTGTCTCCGACTCAG |
| | MID1 | ACGAGTGCGT |
| 266 | StrepPGMF3106e | CCATCTCATCCCTGCGTGTCTCCGACTCAG |
| | MID1 | ACGAGTGCGT |
| 267 | StrepPGMR3366a | CCTCTCTATGGGCAGTCGGTGAT |
| 268 | StrepPGMR3366b | CCTCTCTATGGGCAGTCGGTGAT |
| 269 | StrepPGMR3366c | CCTCTCTATGGGCAGTCGGTGAT |
| 270 | StrepPGMR3366d | CCTCTCTATGGGCAGTCGGTGAT |
| 271 | StrepPGMR3366e | CCTCTCTATGGGCAGTCGGTGAT |
| 272 | StrepPGMR3366f | CCTCTCTATGGGCAGTCGGTGAT |

TABLE 6
Staphylococcus Sequences

| | | | |
|---|---|---|---|
| SEQ | | | |
| ID | | | |
| NO: | Name | Sequence | Description |

Staphylococcus Fragment 1

| | | | |
|---|---|---|---|
| 273 | StaphF44a | GAAACTACGCGAGAATTTCAG | aureus, |
| | | AAG | lugdunensis |
| 274 | StaphF44b | GAAATTACGCGAGAATTTCAG | epidermidis, |
| | | AAG | capitis |
| 275 | StaphF44c | GAAATTATGCGAGAATTTCAG | haemolyticus |
| | | AAG | |
| 276 | StaphF44d | GAAACTATGCGAGAATTTCAG | saprophyticus |
| | | AGG | |
| 277 | StaphR278a | CGAAGAGGTGCAGCATAAGTA | |
| | | G | |
| 278 | StaphR278b | CGTAATGGTGCCGCGTATGTT | intermedius |
| | | G | |
| 279 | StaphR278c | CGTAGAGGTGCAGAATACGTT | saprophyticus |
| | | G | |
| 280 | StaphF18a | CCAATATGGAAGACATCGTAA | |
| | | ACG | |

Staphylococcus Fragment 2

| | | | |
|---|---|---|---|
| 281 | StaphF1251a | CCAATTCCGTATCGGTTTATC | |
| 282 | StaphF1251b | CCAATTCCGTATTGGTTTATC | lugdunensis, |
| | | | saprophyticus |
| 283 | StaphR1505a | ACTTCCATTTGAGCACGTTC | |
| 284 | StaphR1505b | ACTTCCATTTGGGCACGTTC | caprae |
| 285 | StaphR1505c | ACTTCCATTTGTGCACGTTC | lugdunensis |

Staphylococcus Fragment 3

| | | | |
|---|---|---|---|
| 286 | StaphF1484a | GTGAACGTGCTCAAATGGAAG | |
| 287 | StaphF1484b | GTGAACGTGCCCAAATGGAAG | caprae |
| 288 | StaphF1484c | GTGAACGTGCACAAATGGAAG | lugdunensis |
| 289 | StaphR1715a | ACATAGCTATCTTCTTCATCA | |
| | | GC | |
| 290 | StaphR1715b | ACGTAACTATCCTCTTCATCA | epidermidis |
| | | GC | |
| 291 | StaphR1715c | ACATAGCTATCCTCTTCATCA | epidermidis |
| | | GC | |
| 292 | StaphR1715d | ACATAGCTATCTTCTTCGTCA | aureus |
| | | GC | |
| 293 | StaphR1715e | ACATAACTGTCTTCTTCATCA | lugdunensis |
| | | GC | |

Staphylococcus Fragment 4

| | | | |
|---|---|---|---|
| 294 | StaphF3224a | TCGGTGAGATGGAGGTATGG | |
| 295 | StaphF3224b | TCGGTGAGATGGAAGTATGG | lugdunensis |
| 296 | StaphF3224c | TCGGTGAAATGGAAGTATGG | saprophyticus |
| 297 | StaphR3388a | CTCGGAATGATTCTGGAACAC | |
| 298 | StaphR3388b | CTCGGAATGATTCAGGAACAC | intermedius, |
| | | | capitis, |
| | | | lugdunensis, |
| | | | saprophyticus |

TABLE 7
Burkholderia Sequences

| SEQ | | |
|---|---|---|
| ID | | |
| NO: | Name | Sequence |
| 299 | BUR3 | GAAGAAGCAGTTCGGCAA |
| 300 | BUR4 | GAGTCGATGACGATCAT |
| 301 | recAF1 | CCACGCTCACGCTGCAGG |
| 302 | recAR1 | CGAGCCCGAGCGCACCAG |
| 303 | recAF2 | CGAAGGCGAGATGGGCG |
| 304 | recAR2 | TCGAGACGCACCGACG |
| 305 | recAF3 | GTGCAGGCGAAGATCGTCG |
| 306 | recAR3 | CCATCGCCTCGGCTTCG |

TABLE 8
Enterococcus Sequences

| SEQ | | |
|---|---|---|
| ID | | |
| NO: | Name | Sequence |
| 307 | tufF1 | GGCGGACGTCACACTCCATTC |
| 308 | tufR1 | CCGTCTTCGATAGCGATTGGGTGG |
| 309 | tufF2 | GGTTGCTCGTGAAGACATCCAAC |
| 310 | tufR2 | CACCAGTAACGTCTGTTGTACGG |
| 311 | tufF3 | CAGGCGATGATGTTCCAGTTATCGC |
| 312 | tufR3 | GTAGCAACAGTACCACGTCCAGTG |

TABLE 9
Pseudomonas Sequences

| SEQ | | |
|---|---|---|
| ID | | |
| NO: | Name | Sequence |
| 313 | APrU 34 | TGTAAAACGACGGCCAGTGCNGGR |
| | | TCYTTYTCYTGRCA |
| 314 | M13 (21) 34 | TGTAAAACGACGGCCAGT |
| 315 | UP1E 34 | CAGGAAACAGCTATGACCAYGSNG |
| | | GNGGNAARTTYRA |
| 316 | M13R 34 | CAGGAAACAGCTATGACC |
| 317 | gyrbF1 | CAGCTGGGACATCCTGGCC |
| 318 | gyrbR1 | TGAGGGATGTTGTTGGTAAAGCAC |
| 319 | gyrbF2 | GTGCTTTACCAACAACATCCCTCA |
| 320 | gyrbR2 | TGTCTTTGGTCTGGGAGCTGAAC |

TABLE 10
IDT Label Sequences

| SEQ | | |
|---|---|---|
| ID | | |
| NO: | Name | Sequence |
| 321 | Br2-F-MID2 | CCATCTCATCCCTGCGTGTCTCCGACTCAG |
| | | ACGCTCGACAAGYGGCGIACGGGTGAGTAA |
| 322 | Br2-F-MID3 | CCATCTCATCCCTGCGTGTCTCCGACTCAG |
| | | AGACGCACTCAGYGGCGIACGGGTGAGTAA |
| 323 | Br2-F-MID4 | CCATCTCATCCCTGCGTGTCTCCGACTCAG |
| | | AGCACTGTAGAGYGGCGIACGGGTGAGTAA |
| 324 | Br2-F-MID5 | CCATCTCATCCCTGCGTGTCTCCGACTCAG |
| | | ATCAGACACGAGYGGCGIACGGGTGAGTAA |
| 325 | Br2-F-MID6 | CCATCTCATCCCTGCGTGTCTCCGACTCAG |
| | | ATATCGCGAGAGYGGCGIACGGGTGAGTAA |
| 326 | Br2-F-MID7 | CCATCTCATCCCTGCGTGTCTCCGACTCAG |
| | | CGTGTCTCTAAGYGGCGIACGGGTGAGTAA |
| 327 | Br2-F-MID8 | CCATCTCATCCCTGCGTGTCTCCGACTCAG |
| | | CTCGCGTGTCAGYGGCGIACGGGTGAGTAA |
| 328 | Br3-F-MID2 | CCATCTCATCCCTGCGTGTCTCCGACTCAG |
| | | ACGCTCGACAACTCCTACGGGAGGCAGCAG |
| 329 | Br3-F-MID3 | CCATCTCATCCCTGCGTGTCTCCGACTCAG |
| | | AGACGCACTCACTCCTACGGGAGGCAGCAG |
| 330 | Br3-F-MID4 | CCATCTCATCCCTGCGTGTCTCCGACTCAG |
| | | AGCACTGTAGACTCCTACGGGAGGCAGCAG |
| 331 | Br3-F-MID5 | CCATCTCATCCCTGCGTGTCTCCGACTCAG |
| | | ATCAGACACGACTCCTACGGGAGGCAGCAG |
| 332 | Br3-F-MID6 | CCATCTCATCCCTGCGTGTCTCCGACTCAG |
| | | ATATCGCGAGACTCCTACGGGAGGCAGCAG |
| 333 | Br3-F-MID7 | CCATCTCATCCCTGCGTGTCTCCGACTCAG |
| | | CGTGTCTCTAACTCCTACGGGAGGCAGCAG |
| 334 | Br3-F-MID8 | CCATCTCATCCCTGCGTGTCTCCGACTCAG |
| | | ACGAGTGCGTACTCCTACGGGAGGCAGCAG |

The following examples serve to illustrate the present invention. The examples are in no way intended to limit the scope of the invention.

### EXAMPLE 1: Direct Detection Using Primers With Adapter Sequence and Target Specific Sequence

A biological sample is obtained from a human individual and nucleic acid extracted using the MagNA Pure LC instrument (Roche Molecular Diagnostics, Germany). A post-extraction step is performed to remove human nucleic acid from the sample.

The remaining nucleic acid from the sample is amplified using universal 16S rDNA and ITS rDNA primers. The amplification is performed using PCR. The resulting amplified nucleic acid is then amplified again using PCR with bacterial or fungal specific DNA oligonucleotide primer pairs.

Next, primer pairs comprising both a target specific sequence (e.g., specific for a particular sequence within a microbial gene) and an adapter sequence are used to perform a third amplification process. The forward and reverse primers in the primer pairs contain different adapter sequences. The primers can optionally include a MID. This process attaches the adapter sequences to the microbial nucleic acid.

The amplicons are then sequenced using a high throughput, massively parallel platform to identify the nucleic acid sequence of the microbial agent(s) in the sample. The sequences are compared against a BLAST of the rDNA targets to identify the specific microbial agent(s) present in the sample.

### EXAMPLE 2: Direct Detection Using Primers With Adapter Sequence and Multiplex Identifier

A biological sample is obtained from a human individual and nucleic acid extracted using the MagNA Pure LC instrument (Roche Molecular Diagnostics, Germany). A post-extraction step is performed to remove human nucleic acid from the sample.

The remaining nucleic acid from the sample is amplified using universal 16S rDNA and ITS rDNA primers. The amplification is performed using PCR. The resulting amplified nucleic acid is then amplified again using PCR with bacterial or fungal specific DNA oligonucleotide primer pairs comprising a target specific sequence and a tag.

Next, primer pairs comprising an adapter sequence and a MID are used to perform a third amplification process, in which the MID hybridizes to the tag from the second amplification process. The forward and reverse primers in the primer pairs contain different adapter sequences. This process attaches the adapter sequence to the microbial nucleic acid.

The amplicons are then sequenced using a high throughput, massively parallel platform to identify the nucleic acid sequence of the microbial agent(s) in the sample. The sequences are compared against a BLAST of the rDNA targets to identify the specific microbial agent(s) present in the sample.

Alternatively, bi-directional sequencing can be performed, in which the forward and reverse primers in each primer pair have the opposite adapter sequence attached thereto.

### EXAMPLE 3: Direct Detection Using Enzyme Ligation To Attach Adapter

A biological sample is obtained from a human individual and nucleic acid extracted using the MagNA Pure LC instrument (Roche Molecular Diagnostics, Germany). A post-extraction step is performed to remove human nucleic acid from the sample.

The remaining nucleic acid from the sample is amplified using universal 16S rDNA and ITS rDNA primers. The amplification is performed using PCR. The resulting amplified nucleic acid is then amplified again using PCR with bacterial or fungal specific DNA oligonucleotide primer pairs.

Next, enzyme ligation is performed to attached a nucleotide comprising an adapter sequence and MID to the microbial nucleic acid. The resulting adapter-tagged microbial nucleic acid is then amplified using a primer pair to produce amplicons.

The amplicons are then sequenced using a high throughput, massively parallel platform to identify the nucleic acid sequence of the microbial agent(s) in the sample. The sequences are compared against a BLAST of the rDNA targets to identify the specific microbial agent(s) present in the sample.

### EXAMPLE 4: Direct Identification of Different Microbial Species in Polymicrobial Samples

A ~459 bp segment of the V3-V4 bacterial 16s rrna gene was amplified with target-specific PCR primers with 5′ overhang adapters. The amplification mix contained the following ingredients in sufficient volume for a quarter plate and a half plate.

| Reagents | x1 (uL) | x30 (uL) | x60 (uL) |
|---|---|---|---|
| 16Sv3v4-F Primer (1 uM) | 5 | 150 | 300 |
| 16Sv3v4-R Primer (1 uM) | 5 | 150 | 300 |
| 2X KAPA HiFi HotStart | 12.5 | 375 | 750 |
| Ready Mix | | | |
| Total | 22.5 | 675 | 1,350 |

Index sequences and adapters were ligated to the 5′ and 3′ ends of the amplicons to allow for paired end sequencing. The library derived from 15 samples was normalized and pooled, and loaded onto a MiSeq® sequencer for clustering and paired-end sequencing with the 250 bp paired end sequencing chemistry and a nano-flow cell.

Paired-end reads were merged and quality-filtered. Sequences were dereplicated, singletons were discarded, and then sequences clustered into centroids with a radius of 2%. Operational taxonomic units (OTUs) constructed from the centroids for each sample were searched against the Living Tree Program database release 111, available at http://www.arb-silva.de/projects/living-tree/and/or the NCBI 16S rrna sequence database. Species identifications and the relative abundance of each identified species in the samples tested were tabulated.

### Results

### Sequence Metrics

421,105 raw reads were obtained, 368,337 reads passed the quality filtering stage for a PF rate of 87.5%, 93% of reads had a median Q value >Q30. The read distribution was normally distributed between the 15 samples in the pooled library with 4.1%±1.9% (1 SD) reads per sample. The negative control did not have an appreciable number of detectable reads (Table 11).

The vast majority of merged paired end reads produced full length amplicon sequence of 465 bp, or 427 bp after the target-specific PCR primers were trimmed.

TABLE 11
Reads and Operational Taxonomies Units (OTUs) per Sample

| | | % of | reads | |
|---|---|---|---|---|
| | | PF | clustered | OTUs |
| Sample | Description | reads | in OTUs | (>0.5%) |
| M1 | mixed organisms | 4.37 | 13681 | 7 |
| M2 | mixed organisms | 4.05 | 11764 | 14 |
| M3 | mixed organisms | 6.71 | 19967 | 8 |
| M4 | mixed organisms | 3.65 | 10087 | 11 |
| M5 | mixed organisms | 7.77 | 19821 | 8 |
| M6 | mixed organisms | 3.30 | 7232 | 6 |
| S1 | pure sample | 5.55 | 19673 | 1 |
| S2 | pure sample | 2.51 | 8874 | 1 |
| S3 | pure sample | 4.39 | 15577 | 2 |
| S4 | pure sample | 2.27 | 8073 | 1 |
| S5 | pure sample | 6.78 | 23824 | 1 |
| S6 | pure sample | 3.05 | 10899 | 1 |
| P1 | patient sample | 2.78 | 9731 | 1 |
| P2 | patient sample | 1.46 | 5147 | 1 |
| P3 | patient sample | 2.21 | 7705 | 1 |
| NEG | Negative ctrl | 0.007 | NA | NA |

Organism Identification is shown in Table 12 below:

TABLE 12
Identification of Pure (S1-S6) and Patient (P1-P3) Samples

| | | Align | | % of | Reads | |
|---|---|---|---|---|---|---|
| Sample | Identification | (bp) | Match | Reads | (N) | Experimental Input |
| P1_S13 | Pseudomonas_aeruginosa | 425 | 100% | 100% | 9731 | Pseudomonas aeruginosa |
| P2_S14 | Neisseria_sicca | 425 | 99.5% | 100% | 5147 | Neisseria sicca |
| P3_S15 | Bacillus_cereus | 425 | 100% | 100% | 7705 | Bacillus cereus |
| S1_S7 | Neisseria_lactamica | 425 | 100% | 100% | 19673 | Neisseria lactamica |
| S2_S8 | Acinetobacter_baumannii | 425 | 100% | 100% | 8874 | Acinetobacter baumannii |
| S3_S9 | Salmonella_enterica | 425 | 100% | 69.0% | 10753 | Salmonella cholereasuis |
| S3_S9 | Enterobacter_cloacae | 425 | 99.5% | 31.0% | 4824 | |
| S4_S10 | E. fergusonii/E. coli/Shigella | 425 | 100% | 100% | 8073 | Shigella sonnei |
| S5_S11 | Bordetella_parapertussis | 425 | 100% | 100% | 23824 | Bordetella parapertussis |
| S6_S12 | E. fergusonii/E. coli/Shigella | 425 | 100% | 100% | 10899 | E. coli |

The bioinformatic pipeline successfully identified the input species in the 6 pure samples and 3 patient samples (Table 12). Sample 3 appeared to contain a mixture of two species at a 2:1 ratio. The origin of the second species (E. cloacae) is not known.

Table 13 below demonstrates successful recovery of most input species from the mixed samples. E. cloacae, used in samples M1 and M2, appeared as a contaminant in samples M3 and M5.

TABLE 13
Identification of Six Mixed Bacterial Samples

| Sample | OTUId | Reads | Pct | Identity | Input | |
|---|---|---|---|---|---|---|
| M1_S1 | OTU_1 | 5580 | 40.8% | Acinetobacter_baumannii | Acinetobacter baumannii | |
| M1_S1 | OTU_2 | 2480 | 18.1% | E. fergusonii/E. coli/Shigella | Escherichia coli | Shigella sonnei |
| M1_S1 | OTU_3 | 2047 | 15.0% | Enterobacter_aerogenes | Enterobacter aerogenes | |
| M1_S1 | OTU_5 | 1614 | 11.8% | Enterobacter_cloacae | Enterobacter cloacae | |
| M1_S1 | OTU_4 | 861 | 6.3% | Bordetella_pertussis | Bordetella pertussis | |
| M1_S1 | OTU_6 | 573 | 4.2% | Salmonella_enterica | Salmonella choleraesuis | |
| M1_S1 | OTU_7 | 518 | 3.8% | Klebsiella_variicola | Klebsiella pneumoniae | |
| | | | | | Nocardia farcinica | |
| M2_S2 | OTU_1 | 3984 | 33.9% | Staphylococcus_epidermidis/capitis | Staphylococcus epidermidis | Staphylococcus aureus |
| M2_S2 | OTU_15 | 1121 | 9.5% | Staphylococcus_saprophyticus | Staphylococcus saprophyticus | |
| M2_S2 | OTU_3 | 1058 | 9.0% | Streptococcus_pyogenes | Strep pyogenes group a | |
| M2_S2 | OTU_2 | 1032 | 8.8% | Acinetobacter_baumannii | Acinetobacter baumannii | |
| M2_S2 | OTU_5 | 934 | 7.9% | E. fergusonii/E. coli/Shigella | Shigella sonnei | Escherichia coli |
| M2_S2 | OTU_6 | 807 | 6.9% | Streptococcus_agalactiae | Strep agalactiae group b | |
| M2_S2 | OTU_4 | 742 | 6.3% | Neisseria_lactamica | Neisseria lactamica | |
| M2_S2 | OTU_7 | 574 | 4.9% | Proteus_mirabilis | Proteus mirabilis | |
| M2_S2 | OTU_8 | 416 | 3.5% | Enterobacter_aerogenes | Enterobacter aerogenes | |
| M2_S2 | OTU_9 | 296 | 2.5% | Enterobacter_cloacae | Enterobacter cloacae | |
| M2_S2 | OTU 11 | 274 | 2.3% | Haemophilus_aegyptius | Haemophilus influenzae | |
| M2_S2 | OTU_12 | 179 | 1.5% | Bordetella_pertussis | Bordetella parapertussis | Bordetella pertussis |
| M2_S2 | OTU_10 | 166 | 1.4% | Pseudomonas_aeruginosa | Pseudomonas aeruginosa | |
| M2_S2 | OTU_13 | 108 | 0.9% | Salmonella_enterica | Salmonella choleraesuis | |
| M2_S2 | OTU_14 | 73 | 0.6% | Klebsiella_variicola | Klebsiella pneumoniae | |
| | | | | | Ochrobactrum anthropi | |
| | | | | | Camphylobacter jejuni | |
| M3_S3 | OTU_1 | 7103 | 35.6% | Staphylococcus_capitis | Staphylococcus aureus | |
| | OTU_6 | 4117 | 20.6% | Staphylococcus_saprophyticus | Staphylococcus saprophyticus | |
| | OTU_2 | 3380 | 16.9% | Proteus_mirabilis | Proteus mirabilis | |
| | OTU_3 | 2082 | 10.4% | Streptococcus_agalactiae | Strep agalactiae group b | |
| | OTU_4 | 1396 | 7.0% | E. fergusonii/E. coli/Shigella | Escherichia coli | |
| | OTU_5 | 1046 | 5.2% | Pseudomonas_aeruginosa | Pseudomonas aeruginosa | |
| | OTU_7 | 574 | 2.9% | Salmonella_enterica | Salmonella choleraesuis | |
| | OTU_8 | 269 | 1.3% | Enterobacter_cloacae | | |
| M4_S4 | OTU_1 | 3999 | 39.6% | Staphylococcus_epidermidis/capitis | Staphylococcus epidermidis | |
| M4_S4 | OTU_2 | 1677 | 16.6% | Streptococcus_pyogenes | Strep pyogenes group a | |
| M4_S4 | OTU_3 | 1215 | 12.0% | Acinetobacter_baumannii | Acinetobacter baumannii | |
| M4_S4 | OTU_4 | 1069 | 10.6% | Neisseria_lactamica | Neisseria lactamica | |
| M4_S4 | OTU_5 | 533 | 5.3% | Enterobacter_aerogenes | Enterobacter aerogenes | |
| M4_S4 | OTU_6 | 490 | 4.9% | Haemophilus_aegyptius | Haemophilus influenzae | |
| M4_S4 | OTU_7 | 369 | 3.7% | Enterobacter_cloacae | Enterobacter cloacae | |
| M4_S4 | OTU_8 | 240 | 2.4% | Bordetella_pertussis | Bordetella parapertussis | Bordetella pertussis |
| M4_S4 | OTU_10 | 222 | 2.2% | E. fergusonii/E. coli/Shigella | Shigella sonnei | |
| M4_S4 | OTU_9 | 137 | 1.4% | Salmonella_enterica | Salmonella choleraesuis | |
| M4_S4 | OTU_11 | 132 | 1.3% | Klebsiella_variicola | Klebsiella pneumoniae | |
| | | | | | Nocardia farcinica | |
| | | | | | Ochrobactrum anthropi | |
| | | | | | Camphylobacter jejuni | |
| M5_S5 | OTU_1 | 6110 | 437.7% | Streptococcus_pyogenes | Strep pyogenes group a | |
| M5_S5 | OTU_2 | 4336 | 310.6% | Acinetobacter_baumannii | Acinetobacter baumannii | |
| M5_S5 | OTU_3 | 3311 | 237.2% | Neisseria_lactamica | Neisseria lactamica | |
| M5_S5 | OTU_4 | 3151 | 225.7% | Proteus_mirabilis | Proteus mirabilis | |
| M5_S5 | OTU_5 | 1526 | 109.3% | Haemophilus_aegyptius | Haemophilus influenzae | |
| M5_S5 | OTU_6 | 720 | 51.6% | E. fergusonii/E. coli/Shigella | Shigella sonnei | |
| M5_S5 | OTU_7 | 453 | 32.4% | Salmonella_enterica | Salmonella choleraesuis | |
| M5_S5 | OTU_8 | 207 | 14.8% | Enterobacter_cloacae | | |
| | | | | | Nocardia farcinica | |
| | | | | | Corynebacterium | |
| M6_S6 | OTU_3 | 1697 | 23.5% | Enterobacter_aerogenes | Enterobacter aerogenes | |
| M6_S6 | OTU_4 | 1546 | 21.4% | Bordetella_pertussis | Bordetella pertussis | Bordetella parapertussis |
| M6_S6 | OTU_5 | 1206 | 16.7% | Enterobacter_cloacae | Enterobacter cloacae | |
| M6_S6 | OTU_2 | 1204 | 16.6% | E. fergusonii/E. coli/Shigella | Escherichia coli | |
| M6_S6 | OTU_1 | 1131 | 15.6% | Pseudomonas_aeruginosa | Pseudomonas aeruginosa | |
| M6_S6 | OTU_6 | 448 | 6.2% | Klebsiella_variicola | Klebsiella pneumoniae | |
| | | | | | Camphylobacter jejuni | |

These results demonstrate that the methods of the present application, which generate high quality paired-end sequence reads for sequence fragments of short length (in this case a 427 bp was used), accurately identify bacterial species in polymicrobial samples through rDNA amplification and sequencing.

### EXAMPLE 5: Direct Identification of Different Microbial Species in Polymicrobial Samples from Subjects Affected by Infections Difficult to Diagnose

Biological fluids, including urine, sputum, vaginal fluid, sperm, blood and synovial fluid are collected from subjects affected by infections that are difficult to diagnose. The subjects are affected by chronic wound infections, lung infections, urinary tract infections, vaginal infections or infections of otherwise sterile body sites or of prosthetic implants. The samples are directly analyzed for the presence of gram-positive and gram-negative bacterial species without the need for culturing the bacterial colonies.

### Results

Organisms that constitute 10% or more of a mixed population of three or more bacterial species that are present in the fluid sample are detected by 16s rDNA as described in Example 4 above.

1-20. (canceled)

21. A kit for determining the presence of a microbial agent in a sample without culturing the microbial agent, comprising at least one primer pair selected from the group consisting of SEQ ID NOs: 89-335, wherein the at least one primer pair amplifies a target sequence of bacterial 16S rDNA, fungal ITS rDNA, Mycobacterium rpoB, Staphylococcus rpoB, Streptococcus rpoB, Burkholderia recA, Enterococcus tuf, or Pseudomonas gvrB.

22. The kit of claim 21, wherein the at least one primer pair amplifies at least one target sequence of bacterial 16S rDNA, and comprises a forward primer and a reverse primer selected from SEQ ID NOs: 89-118.

23. The kit of claim 21, wherein the at least one primer pair amplifies at least one target sequence of fungal ITS rDNA, and comprises a forward primer and a reverse primer selected from SEQ ID NOs: 119-128.

24. The kit of claim 21, wherein the at least one primer pair amplifies at least one target sequence of Mycobacterium rpoB, and comprises a forward primer and a reverse primer selected from SEQ ID NOs: 139-152.

25. The kit of claim 21, wherein the at least one primer pair amplifies at least one target sequence of Streptococcus rpoB, and comprises a forward primer and a reverse primer selected from SEQ ID NOs: 181-233.

26. The kit of claim 21, wherein the at least one primer pair amplifies at least one target sequence of Staphylococcus rpoB, and comprises a forward primer and a reverse primer selected from SEQ ID NOs: 273-298.

27. The kit of claim 21, wherein the at least one primer pair amplifies at least one target sequence of Burkholderia recA, and comprises a forward primer and a reverse primer selected from SEQ ID NOs: 299-306.

28. The kit of claim 21, wherein the at least one primer pair amplifies at least one target sequence of Enterococcus tuf, and comprises a forward primer and a reverse primer selected from SEQ ID NOs: 307-312.

29. The kit of claim 21, wherein the at least one primer pair amplifies at least one target sequence of Pseudomonas gvrB, and comprises a forward primer and a reverse primer selected from SEQ ID NOs: 313-320.

30. The kit of claim 21, wherein one or more of the primers comprises an adapter sequence selected from the group consisting of SEQ ID NO: 1 and SEQ ID NO: 2.

31. The kit of claim 21, wherein the primers further comprise a multiplex identifier sequence.

32. The kit of claim 31, wherein the primers further comprise a tag sequence selected from the group consisting of SEQ ID NO: 3 and SEQ ID NO: 4.

33. A kit for determining the presence of a microbial agent in a sample without culturing the microbial agent, comprising a primer pair that amplifies at least one target sequence of bacterial 16S rDNA, wherein the primer pair comprises a forward primer selected from among SEQ ID NOs: 89-95, 98-102, 104-110, or 113-117 and a reverse primer selected from among SEQ ID NOs: 96, 97, 103, 111, 112, or 118.

34. The kit of claim 33, wherein one or more of the primers comprises an adapter sequence selected from the group consisting of SEQ ID NO: 1 and SEQ ID NO: 2.

35. The kit of claim 33, wherein the primers further comprise a multiplex identifier sequence.

36. The kit of claim 35, wherein the primers further comprise a tag sequence selected from the group consisting of SEQ ID NO: 3 and SEQ ID NO: 4.

37. A kit for determining the presence of a microbial agent in a sample without culturing the microbial agent, comprising a primer pair that amplifies at least one target sequence of fungal ITS rDNA, wherein the primer pair comprises a forward primer selected from among SEQ ID NOs: 119, 121, 123, 125, 127, 129, 131, 133, 135, 137, 335, or 337 and a reverse primer selected from among SEQ ID NOs: 120, 122, 124, 126, 128, 130, 132, 134, 136, 138, 336, or 338.

38. The kit of claim 37, wherein one or more of the primers comprises an adapter sequence selected from the group consisting of SEQ ID NO: 1 and SEQ ID NO: 2.

39. The kit of claim 37, wherein the primers further comprise a multiplex identifier sequence.

40. The kit of claim 39, wherein the primers further comprise a tag sequence selected from the group consisting of SEQ ID NO: 3 and SEQ ID NO: 4.


SEQUENCE LISTING (338 sequences)

SEQ ID NO: 1 - DNA, 30 bp, synthetic construct
  misc_feature 1..30: note = Description of Artificial Sequence: Syntheticprimer
  source 1..30: mol_type = other DNA
ccatctcatccctgcgtgtctccgactcag

SEQ ID NO: 2 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 3 - DNA, 22 bp, synthetic construct
  misc_feature 1..22: note = Description of Artificial Sequence: Syntheticprimer
  source 1..22: mol_type = other DNA
acactgacgacatggttctaca

SEQ ID NO: 4 - DNA, 22 bp, synthetic construct
  misc_feature 1..22: note = Description of Artificial Sequence: Syntheticprimer
  source 1..22: mol_type = other DNA
tacggtagcagagacttggtct

SEQ ID NO: 5 - DNA, 10 bp, synthetic construct
  misc_feature 1..10: note = Description of Artificial Sequence: Syntheticprimer
  source 1..10: mol_type = other DNA
acgagtgcgt

SEQ ID NO: 6 - DNA, 10 bp, synthetic construct
  misc_feature 1..10: note = Description of Artificial Sequence: Syntheticprimer
  source 1..10: mol_type = other DNA
acgctcgaca

SEQ ID NO: 7 - DNA, 10 bp, synthetic construct
  misc_feature 1..10: note = Description of Artificial Sequence: Syntheticprimer
  source 1..10: mol_type = other DNA
agacgcactc

SEQ ID NO: 8 - DNA, 10 bp, synthetic construct
  misc_feature 1..10: note = Description of Artificial Sequence: Syntheticprimer
  source 1..10: mol_type = other DNA
agcactgtag

SEQ ID NO: 9 - DNA, 10 bp, synthetic construct
  misc_feature 1..10: note = Description of Artificial Sequence: Syntheticprimer
  source 1..10: mol_type = other DNA
atcagacacg

SEQ ID NO: 10 - DNA, 10 bp, synthetic construct
  misc_feature 1..10: note = Description of Artificial Sequence: Syntheticprimer
  source 1..10: mol_type = other DNA
atatcgcgag

SEQ ID NO: 11 - DNA, 10 bp, synthetic construct
  misc_feature 1..10: note = Description of Artificial Sequence: Syntheticprimer
  source 1..10: mol_type = other DNA
cgtgtctcta

SEQ ID NO: 12 - DNA, 10 bp, synthetic construct
  misc_feature 1..10: note = Description of Artificial Sequence: Syntheticprimer
  source 1..10: mol_type = other DNA
ctcgcgtgtc

SEQ ID NO: 13 - DNA, 10 bp, synthetic construct
  misc_feature 1..10: note = Description of Artificial Sequence: Syntheticprimer
  source 1..10: mol_type = other DNA
tagtatcagc

SEQ ID NO: 14 - DNA, 10 bp, synthetic construct
  misc_feature 1..10: note = Description of Artificial Sequence: Syntheticprimer
  source 1..10: mol_type = other DNA
tctctatgcg

SEQ ID NO: 15 - DNA, 10 bp, synthetic construct
  misc_feature 1..10: note = Description of Artificial Sequence: Syntheticprimer
  source 1..10: mol_type = other DNA
tgatacgtct

SEQ ID NO: 16 - DNA, 10 bp, synthetic construct
  misc_feature 1..10: note = Description of Artificial Sequence: Syntheticprimer
  source 1..10: mol_type = other DNA
tactgagcta

SEQ ID NO: 17 - DNA, 10 bp, synthetic construct
  misc_feature 1..10: note = Description of Artificial Sequence: Syntheticprimer
  source 1..10: mol_type = other DNA
catagtagtg

SEQ ID NO: 18 - DNA, 10 bp, synthetic construct
  misc_feature 1..10: note = Description of Artificial Sequence: Syntheticprimer
  source 1..10: mol_type = other DNA
cgagagatac

SEQ ID NO: 19 - DNA, 10 bp, synthetic construct
  misc_feature 1..10: note = Description of Artificial Sequence: Syntheticprimer
  source 1..10: mol_type = other DNA
atacgacgta

SEQ ID NO: 20 - DNA, 10 bp, synthetic construct
  misc_feature 1..10: note = Description of Artificial Sequence: Syntheticprimer
  source 1..10: mol_type = other DNA
tcacgtacta

SEQ ID NO: 21 - DNA, 10 bp, synthetic construct
  misc_feature 1..10: note = Description of Artificial Sequence: Syntheticprimer
  source 1..10: mol_type = other DNA
cgtctagtac

SEQ ID NO: 22 - DNA, 10 bp, synthetic construct
  misc_feature 1..10: note = Description of Artificial Sequence: Syntheticprimer
  source 1..10: mol_type = other DNA
tctacgtagc

SEQ ID NO: 23 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagacgagtgcgt

SEQ ID NO: 24 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagacgctcgaca

SEQ ID NO: 25 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagagacgcactc

SEQ ID NO: 26 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagagcactgtag

SEQ ID NO: 27 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagatcagacacg

SEQ ID NO: 28 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagatatcgcgag

SEQ ID NO: 29 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagcgtgtctcta

SEQ ID NO: 30 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagctcgcgtgtc

SEQ ID NO: 31 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagtagtatcagc

SEQ ID NO: 32 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagtctctatgcg

SEQ ID NO: 33 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagtgatacgtct

SEQ ID NO: 34 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagtactgagcta

SEQ ID NO: 35 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagcatagtagtg

SEQ ID NO: 36 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagcgagagatac

SEQ ID NO: 37 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagatacgacgta

SEQ ID NO: 38 - DNA, 40 bp, synthetic construct
  misc_feature 1..40: note = Description of Artificial Sequence: Syntheticprimer
  source 1..40: mol_type = other DNA
ccatctcatccctgcgtgtctccgactcagtcacgtacta

SEQ ID NO: 39 - DNA, 40 bp, synthetic construct
  misc_feature 1..40: note = Description of Artificial Sequence: Syntheticprimer
  source 1..40: mol_type = other DNA
acgcactcgtctgagtcggagacacgcagggatgagatgg

SEQ ID NO: 40 - DNA, 40 bp, synthetic construct
  misc_feature 1..40: note = Description of Artificial Sequence: Syntheticprimer
  source 1..40: mol_type = other DNA
tgtcgagcgtctgagtcggagacacgcagggatgagatgg

SEQ ID NO: 41 - DNA, 40 bp, synthetic construct
  misc_feature 1..40: note = Description of Artificial Sequence: Syntheticprimer
  source 1..40: mol_type = other DNA
gagtgcgtctctgagtcggagacacgcagggatgagatgg

SEQ ID NO: 42 - DNA, 40 bp, synthetic construct
  misc_feature 1..40: note = Description of Artificial Sequence: Syntheticprimer
  source 1..40: mol_type = other DNA
ctacagtgctctgagtcggagacacgcagggatgagatgg

SEQ ID NO: 43 - DNA, 40 bp, synthetic construct
  misc_feature 1..40: note = Description of Artificial Sequence: Syntheticprimer
  source 1..40: mol_type = other DNA
cgtgtctgatctgagtcggagacacgcagggatgagatgg

SEQ ID NO: 44 - DNA, 40 bp, synthetic construct
  misc_feature 1..40: note = Description of Artificial Sequence: Syntheticprimer
  source 1..40: mol_type = other DNA
ctcgcgatatctgagtcggagacacgcagggatgagatgg

SEQ ID NO: 45 - DNA, 40 bp, synthetic construct
  misc_feature 1..40: note = Description of Artificial Sequence: Syntheticprimer
  source 1..40: mol_type = other DNA
tagagacacgctgagtcggagacacgcagggatgagatgg

SEQ ID NO: 46 - DNA, 40 bp, synthetic construct
  misc_feature 1..40: note = Description of Artificial Sequence: Syntheticprimer
  source 1..40: mol_type = other DNA
gacacgcgagctgagtcggagacacgcagggatgagatgg

SEQ ID NO: 47 - DNA, 40 bp, synthetic construct
  misc_feature 1..40: note = Description of Artificial Sequence: Syntheticprimer
  source 1..40: mol_type = other DNA
gctgatactactgagtcggagacacgcagggatgagatgg

SEQ ID NO: 48 - DNA, 40 bp, synthetic construct
  misc_feature 1..40: note = Description of Artificial Sequence: Syntheticprimer
  source 1..40: mol_type = other DNA
cgcatagagactgagtcggagacacgcagggatgagatgg

SEQ ID NO: 49 - DNA, 40 bp, synthetic construct
  misc_feature 1..40: note = Description of Artificial Sequence: Syntheticprimer
  source 1..40: mol_type = other DNA
agacgtatcactgagtcggagacacgcagggatgagatgg

SEQ ID NO: 50 - DNA, 40 bp, synthetic construct
  misc_feature 1..40: note = Description of Artificial Sequence: Syntheticprimer
  source 1..40: mol_type = other DNA
tagctcagtactgagtcggagacacgcagggatgagatgg

SEQ ID NO: 51 - DNA, 40 bp, synthetic construct
  misc_feature 1..40: note = Description of Artificial Sequence: Syntheticprimer
  source 1..40: mol_type = other DNA
cactactatgctgagtcggagacacgcagggatgagatgg

SEQ ID NO: 52 - DNA, 40 bp, synthetic construct
  misc_feature 1..40: note = Description of Artificial Sequence: Syntheticprimer
  source 1..40: mol_type = other DNA
gtatctctcgctgagtcggagacacgcagggatgagatgg

SEQ ID NO: 53 - DNA, 40 bp, synthetic construct
  misc_feature 1..40: note = Description of Artificial Sequence: Syntheticprimer
  source 1..40: mol_type = other DNA
tacgtcgtatctgagtcggagacacgcagggatgagatgg

SEQ ID NO: 54 - DNA, 40 bp, synthetic construct
  misc_feature 1..40: note = Description of Artificial Sequence: Syntheticprimer
  source 1..40: mol_type = other DNA
tagtacgtgactgagtcggagacacgcagggatgagatgg

SEQ ID NO: 55 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagacgagtgcgtacactgacgacatggttctaca

SEQ ID NO: 56 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagacgctcgacaacactgacgacatggttctaca

SEQ ID NO: 57 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagagacgcactcacactgacgacatggttctaca

SEQ ID NO: 58 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagagcactgtagacactgacgacatggttctaca

SEQ ID NO: 59 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagatcagacacgacactgacgacatggttctaca

SEQ ID NO: 60 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagatatcgcgagacactgacgacatggttctaca

SEQ ID NO: 61 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagcgtgtctctaacactgacgacatggttctaca

SEQ ID NO: 62 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagctcgcgtgtcacactgacgacatggttctaca

SEQ ID NO: 63 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtagtatcagcacactgacgacatggttctaca

SEQ ID NO: 64 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtctctatgcgacactgacgacatggttctaca

SEQ ID NO: 65 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtgatacgtctacactgacgacatggttctaca

SEQ ID NO: 66 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtactgagctaacactgacgacatggttctaca

SEQ ID NO: 67 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagcatagtagtgacactgacgacatggttctaca

SEQ ID NO: 68 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagcgagagatacacactgacgacatggttctaca

SEQ ID NO: 69 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagatacgacgtaacactgacgacatggttctaca

SEQ ID NO: 70 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtcacgtactaacactgacgacatggttctaca

SEQ ID NO: 71 - DNA, 45 bp, synthetic construct
  misc_feature 1..45: note = Description of Artificial Sequence: Syntheticprimer
  source 1..45: mol_type = other DNA
cctctctatgggcagtcggtgattacggtagcagagacttggtct

SEQ ID NO: 72 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagacgagtgcgttacggtagcagagacttggtct

SEQ ID NO: 73 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagacgctcgacatacggtagcagagacttggtct

SEQ ID NO: 74 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagagacgcactctacggtagcagagacttggtct

SEQ ID NO: 75 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagagcactgtagtacggtagcagagacttggtct

SEQ ID NO: 76 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagatcagacacgtacggtagcagagacttggtct

SEQ ID NO: 77 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagatatcgcgagtacggtagcagagacttggtct

SEQ ID NO: 78 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagcgtgtctctatacggtagcagagacttggtct

SEQ ID NO: 79 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagctcgcgtgtctacggtagcagagacttggtct

SEQ ID NO: 80 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtagtatcagctacggtagcagagacttggtct

SEQ ID NO: 81 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtctctatgcgtacggtagcagagacttggtct

SEQ ID NO: 82 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtgatacgtcttacggtagcagagacttggtct

SEQ ID NO: 83 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtactgagctatacggtagcagagacttggtct

SEQ ID NO: 84 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagcatagtagtgtacggtagcagagacttggtct

SEQ ID NO: 85 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagcgagagatactacggtagcagagacttggtct

SEQ ID NO: 86 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagatacgacgtatacggtagcagagacttggtct

SEQ ID NO: 87 - DNA, 62 bp, synthetic construct
  misc_feature 1..62: note = Description of Artificial Sequence: Syntheticprimer
  source 1..62: mol_type = other DNA
ccatctcatccctgcgtgtctccgactcagtcacgtactatacggtagcagagacttggtct

SEQ ID NO: 88 - DNA, 45 bp, synthetic construct
  misc_feature 1..45: note = Description of Artificial Sequence: Syntheticprimer
  source 1..45: mol_type = other DNA
cctctctatgggcagtcggtgatacactgacgacatggttctaca

SEQ ID NO: 89 - DNA, 17 bp, synthetic construct
  misc_feature 1..17: note = Description of Artificial Sequence: Syntheticprimer
  source 1..17: mol_type = other DNA
ggcggacgggtgagtaa

SEQ ID NO: 90 - DNA, 17 bp, synthetic construct
  misc_feature 1..17: note = Description of Artificial Sequence: Syntheticprimer
  source 1..17: mol_type = other DNA
ggcgaacgggtgagtaa

SEQ ID NO: 91 - DNA, 17 bp, synthetic construct
  misc_feature 1..17: note = Description of Artificial Sequence: Syntheticprimer
  source 1..17: mol_type = other DNA
ggcgcacgggtgagtaa

SEQ ID NO: 92 - DNA, 17 bp, synthetic construct
  misc_feature 1..17: note = Description of Artificial Sequence: Syntheticprimer
  source 1..17: mol_type = other DNA
ggcggatgggtgagtaa

SEQ ID NO: 93 - DNA, 17 bp, synthetic construct
  misc_feature 1..17: note = Description of Artificial Sequence: Syntheticprimer
  source 1..17: mol_type = other DNA
ggcaaacgggtgagtaa

SEQ ID NO: 94 - DNA, 17 bp, synthetic construct
  misc_feature 1..17: note = Description of Artificial Sequence: Syntheticprimer
  source 1..17: mol_type = other DNA
ggcgaacgggcgagtaa

SEQ ID NO: 95 - DNA, 17 bp, synthetic construct
  misc_feature 1..17: note = Description of Artificial Sequence: Syntheticprimer
  source 1..17: mol_type = other DNA
ggcgaacggctgagtaa

SEQ ID NO: 96 - DNA, 18 bp, synthetic construct
  misc_feature 1..18: note = Description of Artificial Sequence: Syntheticprimer
  source 1..18: mol_type = other DNA
cactgctgcctcccgtag

SEQ ID NO: 97 - DNA, 18 bp, synthetic construct
  misc_feature 1..18: note = Description of Artificial Sequence: Syntheticprimer
  source 1..18: mol_type = other DNA
tactgctgcctcccgtag

SEQ ID NO: 98 - DNA, 20 bp, synthetic construct
  misc_feature 1..20: note = Description of Artificial Sequence: Syntheticprimer
  source 1..20: mol_type = other DNA
gacacggtccagactcctac

SEQ ID NO: 99 - DNA, 20 bp, synthetic construct
  misc_feature 1..20: note = Description of Artificial Sequence: Syntheticprimer
  source 1..20: mol_type = other DNA
gacacggcccagactcctac

SEQ ID NO: 100 - DNA, 20 bp, synthetic construct
  misc_feature 1..20: note = Description of Artificial Sequence: Syntheticprimer
  source 1..20: mol_type = other DNA
gacacggtccaaactcctac

SEQ ID NO: 101 - DNA, 20 bp, synthetic construct
  misc_feature 1..20: note = Description of Artificial Sequence: Syntheticprimer
  source 1..20: mol_type = other DNA
gacacggcccaaactcctac

SEQ ID NO: 102 - DNA, 20 bp, synthetic construct
  misc_feature 1..20: note = Description of Artificial Sequence: Syntheticprimer
  source 1..20: mol_type = other DNA
gatacggcccagactcctac

SEQ ID NO: 103 - DNA, 16 bp, synthetic construct
  misc_feature 1..16: note = Description of Artificial Sequence: Syntheticprimer
  source 1..16: mol_type = other DNA
attaccgcggctgctg

SEQ ID NO: 104 - DNA, 39 bp, synthetic construct
  misc_feature 1..39: note = Description of Artificial Sequence: Syntheticprimer
  source 1..39: mol_type = other DNA
acactgacgacatggttctacaggcggacgggtgagtaa

SEQ ID NO: 105 - DNA, 39 bp, synthetic construct
  misc_feature 1..39: note = Description of Artificial Sequence: Syntheticprimer
  source 1..39: mol_type = other DNA
acactgacgacatggttctacaggcgaacgggtgagtaa

SEQ ID NO: 106 - DNA, 39 bp, synthetic construct
  misc_feature 1..39: note = Description of Artificial Sequence: Syntheticprimer
  source 1..39: mol_type = other DNA
acactgacgacatggttctacaggcgcacgggtgagtaa

SEQ ID NO: 107 - DNA, 39 bp, synthetic construct
  misc_feature 1..39: note = Description of Artificial Sequence: Syntheticprimer
  source 1..39: mol_type = other DNA
acactgacgacatggttctacaggcggatgggtgagtaa

SEQ ID NO: 108 - DNA, 39 bp, synthetic construct
  misc_feature 1..39: note = Description of Artificial Sequence: Syntheticprimer
  source 1..39: mol_type = other DNA
acactgacgacatggttctacaggcaaacgggtgagtaa

SEQ ID NO: 109 - DNA, 39 bp, synthetic construct
  misc_feature 1..39: note = Description of Artificial Sequence: Syntheticprimer
  source 1..39: mol_type = other DNA
acactgacgacatggttctacaggcgaacgggcgagtaa

SEQ ID NO: 110 - DNA, 39 bp, synthetic construct
  misc_feature 1..39: note = Description of Artificial Sequence: Syntheticprimer
  source 1..39: mol_type = other DNA
acactgacgacatggttctacaggcgaacggctgagtaa

SEQ ID NO: 111 - DNA, 40 bp, synthetic construct
  misc_feature 1..40: note = Description of Artificial Sequence: Syntheticprimer
  source 1..40: mol_type = other DNA
tacggtagcagagacttggtctcactgctgcctcccgtag

SEQ ID NO: 112 - DNA, 40 bp, synthetic construct
  misc_feature 1..40: note = Description of Artificial Sequence: Syntheticprimer
  source 1..40: mol_type = other DNA
tacggtagcagagacttggtcttactgctgcctcccgtag

SEQ ID NO: 113 - DNA, 42 bp, synthetic construct
  misc_feature 1..42: note = Description of Artificial Sequence: Syntheticprimer
  source 1..42: mol_type = other DNA
acactgacgacatggttctacagacacggtccagactcctac

SEQ ID NO: 114 - DNA, 42 bp, synthetic construct
  misc_feature 1..42: note = Description of Artificial Sequence: Syntheticprimer
  source 1..42: mol_type = other DNA
acactgacgacatggttctacagacacggcccagactcctac

SEQ ID NO: 115 - DNA, 42 bp, synthetic construct
  misc_feature 1..42: note = Description of Artificial Sequence: Syntheticprimer
  source 1..42: mol_type = other DNA
acactgacgacatggttctacagacacggtccaaactcctac

SEQ ID NO: 116 - DNA, 42 bp, synthetic construct
  misc_feature 1..42: note = Description of Artificial Sequence: Syntheticprimer
  source 1..42: mol_type = other DNA
acactgacgacatggttctacagacacggcccaaactcctac

SEQ ID NO: 117 - DNA, 42 bp, synthetic construct
  misc_feature 1..42: note = Description of Artificial Sequence: Syntheticprimer
  source 1..42: mol_type = other DNA
acactgacgacatggttctacagatacggcccagactcctac

SEQ ID NO: 118 - DNA, 38 bp, synthetic construct
  misc_feature 1..38: note = Description of Artificial Sequence: Syntheticprimer
  source 1..38: mol_type = other DNA
tacggtagcagagacttggtctattaccgcggctgctg

SEQ ID NO: 119 - DNA, 25 bp, synthetic construct
  misc_feature 1..25: note = Description of Artificial Sequence: Syntheticprimer
  source 1..25: mol_type = other DNA
aaactcggtcatttagaggaagtaa

SEQ ID NO: 120 - DNA, 19 bp, synthetic construct
  misc_feature 1..19: note = Description of Artificial Sequence: Syntheticprimer
  source 1..19: mol_type = other DNA
gatgccggaaccaagagat

SEQ ID NO: 121 - DNA, 20 bp, synthetic construct
  misc_feature 1..20: note = Description of Artificial Sequence: Syntheticprimer
  source 1..20: mol_type = other DNA
aacctcccacccgtgtttat

SEQ ID NO: 122 - DNA, 20 bp, synthetic construct
  misc_feature 1..20: note = Description of Artificial Sequence: Syntheticprimer
  source 1..20: mol_type = other DNA
atttcgctgcgttcttcatc

SEQ ID NO: 123 - DNA, 25 bp, synthetic construct
  misc_feature 1..25: note = Description of Artificial Sequence: Syntheticprimer
  source 1..25: mol_type = other DNA
aaactcggtcatttagaggaagtaa

SEQ ID NO: 124 - DNA, 19 bp, synthetic construct
  misc_feature 1..19: note = Description of Artificial Sequence: Syntheticprimer
  source 1..19: mol_type = other DNA
gctgcgttcttcatcgatg

SEQ ID NO: 125 - DNA, 20 bp, synthetic construct
  misc_feature 1..20: note = Description of Artificial Sequence: Syntheticprimer
  source 1..20: mol_type = other DNA
atcgagtctttgaacgcaca

SEQ ID NO: 126 - DNA, 20 bp, synthetic construct
  misc_feature 1..20: note = Description of Artificial Sequence: Syntheticprimer
  source 1..20: mol_type = other DNA
cctacctgatccgaggtcaa

SEQ ID NO: 127 - DNA, 20 bp, synthetic construct
  misc_feature 1..20: note = Description of Artificial Sequence: Syntheticprimer
  source 1..20: mol_type = other DNA
tcgagtctttgaacgcacat

SEQ ID NO: 128 - DNA, 20 bp, synthetic construct
  misc_feature 1..20: note = Description of Artificial Sequence: Syntheticprimer
  source 1..20: mol_type = other DNA
cgggtatccctacctgatcc

SEQ ID NO: 129 - DNA, 65 bp, synthetic construct
  misc_feature 1..65: note = Description of Artificial Sequence: Syntheticprimer
  source 1..65: mol_type = other DNA
ccatctcatccctgcgtgtctccgactcagacgagtgcgtaaactcggtcatttagaggaagtaa

SEQ ID NO: 130 - DNA, 42 bp, synthetic construct
  misc_feature 1..42: note = Description of Artificial Sequence: Syntheticprimer
  source 1..42: mol_type = other DNA
cctctctatgggcagtcggtgatgatgccggaaccaagagat

SEQ ID NO: 131 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagacgagtgcgtaacctcccacccgtgtttat

SEQ ID NO: 132 - DNA, 43 bp, synthetic construct
  misc_feature 1..43: note = Description of Artificial Sequence: Syntheticprimer
  source 1..43: mol_type = other DNA
cctctctatgggcagtcggtgatatttcgctgcgttcttcatc

SEQ ID NO: 133 - DNA, 65 bp, synthetic construct; identical to SEQ ID NO: 129
  misc_feature 1..65: note = Description of Artificial Sequence: Syntheticprimer
  source 1..65: mol_type = other DNA

SEQ ID NO: 134 - DNA, 42 bp, synthetic construct
  misc_feature 1..42: note = Description of Artificial Sequence: Syntheticprimer
  source 1..42: mol_type = other DNA
cctctctatgggcagtcggtgatgctgcgttcttcatcgatg

SEQ ID NO: 135 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagacgagtgcgtatcgagtctttgaacgcaca

SEQ ID NO: 136 - DNA, 43 bp, synthetic construct
  misc_feature 1..43: note = Description of Artificial Sequence: Syntheticprimer
  source 1..43: mol_type = other DNA
cctctctatgggcagtcggtgatcctacctgatccgaggtcaa

SEQ ID NO: 137 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagacgagtgcgttcgagtctttgaacgcacat

SEQ ID NO: 138 - DNA, 43 bp, synthetic construct
  misc_feature 1..43: note = Description of Artificial Sequence: Syntheticprimer
  source 1..43: mol_type = other DNA
cctctctatgggcagtcggtgatcgggtatccctacctgatcc

SEQ ID NO: 139 - DNA, 17 bp, synthetic construct
  misc_feature 1..17: note = Description of Artificial Sequence: Syntheticprimer
  source 1..17: mol_type = other DNA
gcaaggtcaccccgaag

SEQ ID NO: 140 - DNA, 17 bp, synthetic construct
  misc_feature 1..17: note = Description of Artificial Sequence: Syntheticprimer
  source 1..17: mol_type = other DNA
cgatgacgcccttgttg

SEQ ID NO: 141 - DNA, 19 bp, synthetic construct
  misc_feature 1..19: note = Description of Artificial Sequence: Syntheticprimer
  source 1..19: mol_type = other DNA
ggcaaggtcaccccgaagg

SEQ ID NO: 142 - DNA, 18 bp, synthetic construct
  misc_feature 1..18: note = Description of Artificial Sequence: Syntheticprimer
  source 1..18: mol_type = other DNA
aggatcttgccgatgacg

SEQ ID NO: 143 - DNA, 17 bp, synthetic construct
  misc_feature 1..17: note = Description of Artificial Sequence: Syntheticprimer
  source 1..17: mol_type = other DNA
gacgccacggcaacaag

SEQ ID NO: 144 - DNA, 16 bp, synthetic construct
  misc_feature 1..16: note = Description of Artificial Sequence: Syntheticprimer
  source 1..16: mol_type = other DNA
acgccacggcaacaag

SEQ ID NO: 145 - DNA, 22 bp, synthetic construct
  misc_feature 1..22: note = Description of Artificial Sequence: Syntheticprimer
  source 1..22: mol_type = other DNA
caagtggtgcagcttcaggatg

SEQ ID NO: 146 - DNA, 22 bp, synthetic construct
  misc_feature 1..22: note = Description of Artificial Sequence: Syntheticprimer
  source 1..22: mol_type = other DNA
cargtggtgcagcttcakgatg

SEQ ID NO: 147 - DNA, 15 bp, synthetic construct
  misc_feature 1..15: note = Description of Artificial Sequence: Syntheticprimer
  source 1..15: mol_type = other DNA
ggcgccgtcgaacac

SEQ ID NO: 148 - DNA, 15 bp, synthetic construct
  misc_feature 1..15: note = Description of Artificial Sequence: Syntheticprimer
  source 1..15: mol_type = other DNA
ggcrccgtcgaacac

SEQ ID NO: 149 - DNA, 15 bp, synthetic construct
  misc_feature 1..15: note = Description of Artificial Sequence: Syntheticprimer
  source 1..15: mol_type = other DNA
ggcaccgtcgaacac

SEQ ID NO: 150 - DNA, 15 bp, synthetic construct
  misc_feature 1..15: note = Description of Artificial Sequence: Syntheticprimer
  source 1..15: mol_type = other DNA
ggcgccgtcgaacac

SEQ ID NO: 151 - DNA, 16 bp, synthetic construct
  misc_feature 1..16: note = Description of Artificial Sequence: Syntheticprimer
  source 1..16: mol_type = other DNA
caccccggtgttcgac

SEQ ID NO: 152 - DNA, 20 bp, synthetic construct
  misc_feature 1..20: note = Description of Artificial Sequence: Syntheticprimer
  source 1..20: mol_type = other DNA
ctgggtgatcatcgagtacg

SEQ ID NO: 153 - DNA, 47 bp, synthetic construct
  misc_feature 1..47: note = Description of Artificial Sequence: Syntheticprimer
  source 1..47: mol_type = other DNA
ccatctcatccctgcgtgtctccgactcaggcaaggtcaccccgaag

SEQ ID NO: 154 - DNA, 40 bp, synthetic construct
  misc_feature 1..40: note = Description of Artificial Sequence: Syntheticprimer
  source 1..40: mol_type = other DNA
cctctctatgggcagtcggtgatcgatgacgcccttgttg

SEQ ID NO: 155 - DNA, 49 bp, synthetic construct
  misc_feature 1..49: note = Description of Artificial Sequence: Syntheticprimer
  source 1..49: mol_type = other DNA
ccatctcatccctgcgtgtctccgactcagggcaaggtcaccccgaagg

SEQ ID NO: 156 - DNA, 41 bp, synthetic construct
  misc_feature 1..41: note = Description of Artificial Sequence: Syntheticprimer
  source 1..41: mol_type = other DNA
cctctctatgggcagtcggtgataggatcttgccgatgacg

SEQ ID NO: 157 - DNA, 57 bp, synthetic construct
  misc_feature 1..57: note = Description of Artificial Sequence: Syntheticprimer
  source 1..57: mol_type = other DNA
ccatctcatccctgcgtgtctccgactcagacgagtgcgtgcaaggtcaccccgaag

SEQ ID NO: 158 - DNA, 40 bp, synthetic construct
  misc_feature 1..40: note = Description of Artificial Sequence: Syntheticprimer
  source 1..40: mol_type = other DNA
cctctctatgggcagtcggtgatgcaaggtcaccccgaag

SEQ ID NO: 159 - DNA, 47 bp, synthetic construct
  misc_feature 1..47: note = Description of Artificial Sequence: Syntheticprimer
  source 1..47: mol_type = other DNA
ccatctcatccctgcgtgtctccgactcagcgatgacgcccttgttg

SEQ ID NO: 160 - DNA, 42 bp, synthetic construct
  misc_feature 1..42: note = Description of Artificial Sequence: Syntheticprimer
  source 1..42: mol_type = other DNA
cctctctatgggcagtcggtgatggcaaggtcaccccgaagg

SEQ ID NO: 161 - DNA, 48 bp, synthetic construct
  misc_feature 1..48: note = Description of Artificial Sequence: Syntheticprimer
  source 1..48: mol_type = other DNA
ccatctcatccctgcgtgtctccgactcagaggatcttgccgatgacg

SEQ ID NO: 162 - DNA, 47 bp, synthetic construct
  misc_feature 1..47: note = Description of Artificial Sequence: Syntheticprimer
  source 1..47: mol_type = other DNA
ccatctcatccctgcgtgtctccgactcaggacgccacggcaacaag

SEQ ID NO: 163 - DNA, 46 bp, synthetic construct
  misc_feature 1..46: note = Description of Artificial Sequence: Syntheticprimer
  source 1..46: mol_type = other DNA
ccatctcatccctgcgtgtctccgactcagacgccacggcaacaag

SEQ ID NO: 164 - DNA, 45 bp, synthetic construct
  misc_feature 1..45: note = Description of Artificial Sequence: Syntheticprimer
  source 1..45: mol_type = other DNA
cctctctatgggcagtcggtgatcaagtggtgcagcttcaggatg

SEQ ID NO: 165 - DNA, 45 bp, synthetic construct
  misc_feature 1..45: note = Description of Artificial Sequence: Syntheticprimer
  source 1..45: mol_type = other DNA
cctctctatgggcagtcggtgatcargtggtgcagcttcakgatg

SEQ ID NO: 166 - DNA, 38 bp, synthetic construct
  misc_feature 1..38: note = Description of Artificial Sequence: Syntheticprimer
  source 1..38: mol_type = other DNA
cctctctatgggcagtcggtgatggcgccgtcgaacac

SEQ ID NO: 167 - DNA, 38 bp, synthetic construct
  misc_feature 1..38: note = Description of Artificial Sequence: Syntheticprimer
  source 1..38: mol_type = other DNA
cctctctatgggcagtcggtgatggcrccgtcgaacac

SEQ ID NO: 168 - DNA, 57 bp, synthetic construct
  misc_feature 1..57: note = Description of Artificial Sequence: Syntheticprimer
  source 1..57: mol_type = other DNA
ccatctcatccctgcgtgtctccgactcagacgagtgcgtgacgccacggcaacaag

SEQ ID NO: 169 - DNA, 56 bp, synthetic construct
  misc_feature 1..56: note = Description of Artificial Sequence: Syntheticprimer
  source 1..56: mol_type = other DNA
ccatctcatccctgcgtgtctccgactcagacgagtgcgtacgccacggcaacaag

SEQ ID NO: 170 - DNA, 40 bp, synthetic construct
  misc_feature 1..40: note = Description of Artificial Sequence: Syntheticprimer
  source 1..40: mol_type = other DNA
cctctctatgggcagtcggtgatgacgccacggcaacaag

SEQ ID NO: 171 - DNA, 39 bp, synthetic construct
  misc_feature 1..39: note = Description of Artificial Sequence: Syntheticprimer
  source 1..39: mol_type = other DNA
cctctctatgggcagtcggtgatacgccacggcaacaag

SEQ ID NO: 172 - DNA, 52 bp, synthetic construct
  misc_feature 1..52: note = Description of Artificial Sequence: Syntheticprimer
  source 1..52: mol_type = other DNA
ccatctcatccctgcgtgtctccgactcagcaagtggtgcagcttcaggatg

SEQ ID NO: 173 - DNA, 52 bp, synthetic construct
  misc_feature 1..52: note = Description of Artificial Sequence: Syntheticprimer
  source 1..52: mol_type = other DNA
ccatctcatccctgcgtgtctccgactcagcargtggtgcagcttcakgatg

SEQ ID NO: 174 - DNA, 45 bp, synthetic construct
  misc_feature 1..45: note = Description of Artificial Sequence: Syntheticprimer
  source 1..45: mol_type = other DNA
ccatctcatccctgcgtgtctccgactcagggcgccgtcgaacac

SEQ ID NO: 175 - DNA, 45 bp, synthetic construct
  misc_feature 1..45: note = Description of Artificial Sequence: Syntheticprimer
  source 1..45: mol_type = other DNA
ccatctcatccctgcgtgtctccgactcagggcrccgtcgaacac

SEQ ID NO: 176 - DNA, 46 bp, synthetic construct
  misc_feature 1..46: note = Description of Artificial Sequence: Syntheticprimer
  source 1..46: mol_type = other DNA
ccatctcatccctgcgtgtctccgactcagcaccccggtgttcgac

SEQ ID NO: 177 - DNA, 43 bp, synthetic construct
  misc_feature 1..43: note = Description of Artificial Sequence: Syntheticprimer
  source 1..43: mol_type = other DNA
cctctctatgggcagtcggtgatctgggtgatcatcgagtacg

SEQ ID NO: 178 - DNA, 56 bp, synthetic construct
  misc_feature 1..56: note = Description of Artificial Sequence: Syntheticprimer
  source 1..56: mol_type = other DNA
ccatctcatccctgcgtgtctccgactcagacgagtgcgtcaccccggtgttcgac

SEQ ID NO: 179 - DNA, 39 bp, synthetic construct
  misc_feature 1..39: note = Description of Artificial Sequence: Syntheticprimer
  source 1..39: mol_type = other DNA
cctctctatgggcagtcggtgatcaccccggtgttcgac

SEQ ID NO: 180 - DNA, 50 bp, synthetic construct
  misc_feature 1..50: note = Description of Artificial Sequence: Syntheticprimer
  source 1..50: mol_type = other DNA
ccatctcatccctgcgtgtctccgactcagctgggtgatcatcgagtacg

SEQ ID NO: 181 - DNA, 18 bp, synthetic construct
  misc_feature 1..18: note = Description of Artificial Sequence: Syntheticprimer
  source 1..18: mol_type = other DNA
ccttgggacctggtggtt

SEQ ID NO: 182 - DNA, 18 bp, synthetic construct
  misc_feature 1..18: note = Description of Artificial Sequence: Syntheticprimer
  source 1..18: mol_type = other DNA
ccttaggacctggtggtt

SEQ ID NO: 183 - DNA, 19 bp, synthetic construct
  misc_feature 1..19: note = Description of Artificial Sequence: Syntheticprimer
  source 1..19: mol_type = other DNA
gctttaggtcctggtggtt

SEQ ID NO: 184 - DNA, 18 bp, synthetic construct
  misc_feature 1..18: note = Description of Artificial Sequence: Syntheticprimer
  source 1..18: mol_type = other DNA
ccttggggcctggtggtt

SEQ ID NO: 185 - DNA, 18 bp, synthetic construct
  misc_feature 1..18: note = Description of Artificial Sequence: Syntheticprimer
  source 1..18: mol_type = other DNA
ccttagggcctggtggtt

SEQ ID NO: 186 - DNA, 20 bp, synthetic construct
  misc_feature 1..20: note = Description of Artificial Sequence: Syntheticprimer
  source 1..20: mol_type = other DNA
cttcttcgtcggcagtcaac

SEQ ID NO: 187 - DNA, 21 bp, synthetic construct
  misc_feature 1..21: note = Description of Artificial Sequence: Syntheticprimer
  source 1..21: mol_type = other DNA
cttcttcatcagcagtcaacc

SEQ ID NO: 188 - DNA, 20 bp, synthetic construct
  misc_feature 1..20: note = Description of Artificial Sequence: Syntheticprimer
  source 1..20: mol_type = other DNA
cttcttcatcagcagttagc

SEQ ID NO: 189 - DNA, 20 bp, synthetic construct
  misc_feature 1..20: note = Description of Artificial Sequence: Syntheticprimer
  source 1..20: mol_type = other DNA
cttcttcatcagcagtaagc

SEQ ID NO: 190 - DNA, 20 bp, synthetic construct
  misc_feature 1..20: note = Description of Artificial Sequence: Syntheticprimer
  source 1..20: mol_type = other DNA
cttcttcatcagctgtcaac

SEQ ID NO: 191 - DNA, 20 bp, synthetic construct
  misc_feature 1..20: note = Description of Artificial Sequence: Syntheticprimer
  source 1..20: mol_type = other DNA
cttcttcatcggctgtcaac

SEQ ID NO: 192 - DNA, 20 bp, synthetic construct
  misc_feature 1..20: note = Description of Artificial Sequence: Syntheticprimer
  source 1..20: mol_type = other DNA
cttcctcgtcagcggtcaac

SEQ ID NO: 193 - DNA, 20 bp, synthetic construct
  misc_feature 1..20: note = Description of Artificial Sequence: Syntheticprimer
  source 1..20: mol_type = other DNA
cttcttcgtccgctgtcagc

SEQ ID NO: 194 - DNA, 20 bp, synthetic construct
  misc_feature 1..20: note = Description of Artificial Sequence: Syntheticprimer
  source 1..20: mol_type = other DNA
cttcttcatccgctgttagc

SEQ ID NO: 195 - DNA, 20 bp, synthetic construct
  misc_feature 1..20: note = Description of Artificial Sequence: Syntheticprimer
  source 1..20: mol_type = other DNA
tgcgacagcatgtattcctt

SEQ ID NO: 196 - DNA, 20 bp, synthetic construct
  misc_feature 1..20: note = Description of Artificial Sequence: Syntheticprimer
  source 1..20: mol_type = other DNA
cgcaacagcatgtattcctt

SEQ ID NO: 197 - DNA, 20 bp, synthetic construct
  misc_feature 1..20: note = Description of Artificial Sequence: Syntheticprimer
  source 1..20: mol_type = other DNA
tgcaacggcatgtattcctt

SEQ ID NO: 198 - DNA, 20 bp, synthetic construct
  misc_feature 1..20: note = Description of Artificial Sequence: Syntheticprimer
  source 1..20: mol_type = other DNA
ggcaacggcatgtattcctt

SEQ ID NO: 199 - DNA, 21 bp, synthetic construct
  misc_feature 1..21: note = Description of Artificial Sequence: Syntheticprimer
  source 1..21: mol_type = other DNA
tgagtttgaacgacggaattt

SEQ ID NO: 200 - DNA, 21 bp, synthetic construct
  misc_feature 1..21: note = Description of Artificial Sequence: Syntheticprimer
  source 1..21: mol_type = other DNA
tgagttggagcgacggaattt

SEQ ID NO: 201 - DNA, 21 bp, synthetic construct
  misc_feature 1..21: note = Description of Artificial Sequence: Syntheticprimer
  source 1..21: mol_type = other DNA
agagtttgaacggcggaattt

SEQ ID NO: 202 - DNA, 21 bp, synthetic construct
  misc_feature 1..21: note = Description of Artificial Sequence: Syntheticprimer
  source 1..21: mol_type = other DNA
agagttagaacgacggaattt

SEQ ID NO: 203 - DNA, 21 bp, synthetic construct
  misc_feature 1..21: note = Description of Artificial Sequence: Syntheticprimer
  source 1..21: mol_type = other DNA
tgagtttgaacggcggaattt

SEQ ID NO: 204 - DNA, 21 bp, synthetic construct
  misc_feature 1..21: note = Description of Artificial Sequence: Syntheticprimer
  source 1..21: mol_type = other DNA
tgagttagaacgacggaattt

SEQ ID NO: 205 - DNA, 21 bp, synthetic construct
  misc_feature 1..21: note = Description of Artificial Sequence: Syntheticprimer
  source 1..21: mol_type = other DNA
tgagttagaacggcggaattt

SEQ ID NO: 206 - DNA, 21 bp, synthetic construct
  misc_feature 1..21: note = Description of Artificial Sequence: Syntheticprimer
  source 1..21: mol_type = other DNA
tgaacatcggtcaggttatgg

SEQ ID NO: 207 - DNA, 21 bp, synthetic construct
  misc_feature 1..21: note = Description of Artificial Sequence: Syntheticprimer
  source 1..21: mol_type = other DNA
tgaacattggtcaggttatgg

SEQ ID NO: 208 - DNA, 21 bp, synthetic construct
  misc_feature 1..21: note = Description of Artificial Sequence: Syntheticprimer
  source 1..21: mol_type = other DNA
tgaatattggtcaggttatgg

SEQ ID NO: 209 - DNA, 21 bp, synthetic construct
  misc_feature 1..21: note = Description of Artificial Sequence: Syntheticprimer
  source 1..21: mol_type = other DNA
tgaatatcggtcaggttatgg

SEQ ID NO: 210 - DNA, 21 bp, synthetic construct
  misc_feature 1..21: note = Description of Artificial Sequence: Syntheticprimer
  source 1..21: mol_type = other DNA
tgaacatcggacaagttatgg

SEQ ID NO: 211 - DNA, 21 bp, synthetic construct
  misc_feature 1..21: note = Description of Artificial Sequence: Syntheticprimer
  source 1..21: mol_type = other DNA
tgaacattggacaggttatgg

SEQ ID NO: 212 - DNA, 21 bp, synthetic construct
  misc_feature 1..21: note = Description of Artificial Sequence: Syntheticprimer
  source 1..21: mol_type = other DNA
tgaacattgggcaagttatgg

SEQ ID NO: 213 - DNA, 21 bp, synthetic construct
  misc_feature 1..21: note = Description of Artificial Sequence: Syntheticprimer
  source 1..21: mol_type = other DNA
tgaatatcggacaagttatgg

SEQ ID NO: 214 - DNA, 21 bp, synthetic construct
  misc_feature 1..21: note = Description of Artificial Sequence: Syntheticprimer
  source 1..21: mol_type = other DNA
tgaatattggtcaagttatgg

SEQ ID NO: 215 - DNA, 23 bp, synthetic construct
  misc_feature 1..23: note = Description of Artificial Sequence: Syntheticprimer
  source 1..23: mol_type = other DNA
tgaagtttatcatcaaccatgtg

SEQ ID NO: 216 - DNA, 23 bp, synthetic construct
  misc_feature 1..23: note = Description of Artificial Sequence: Syntheticprimer
  source 1..23: mol_type = other DNA
tgcaatttatcatcaaccatgtg

SEQ ID NO: 217 - DNA, 23 bp, synthetic construct
  misc_feature 1..23: note = Description of Artificial Sequence: Syntheticprimer
  source 1..23: mol_type = other DNA
tgcaacttatcatcaaccatgtg

SEQ ID NO: 218 - DNA, 23 bp, synthetic construct
  misc_feature 1..23: note = Description of Artificial Sequence: Syntheticprimer
  source 1..23: mol_type = other DNA
tgaagcttatcatctaccatgtg

SEQ ID NO: 219 - DNA, 23 bp, synthetic construct
  misc_feature 1..23: note = Description of Artificial Sequence: Syntheticprimer
  source 1..23: mol_type = other DNA
tggagtttatcatctaccatgtg

SEQ ID NO: 220 - DNA, 23 bp, synthetic construct
  misc_feature 1..23: note = Description of Artificial Sequence: Syntheticprimer
  source 1..23: mol_type = other DNA
tgaagcttatcatcaaccatgtg

SEQ ID NO: 221 - DNA, 23 bp, synthetic construct
  misc_feature 1..23: note = Description of Artificial Sequence: Syntheticprimer
  source 1..23: mol_type = other DNA
tgcaatttatcgtcaaccatgtg

SEQ ID NO: 222 - DNA, 23 bp, synthetic construct
  misc_feature 1..23: note = Description of Artificial Sequence: Syntheticprimer
  source 1..23: mol_type = other DNA
tggagcttatcatcaaccatgtg

SEQ ID NO: 223 - DNA, 23 bp, synthetic construct
  misc_feature 1..23: note = Description of Artificial Sequence: Syntheticprimer
  source 1..23: mol_type = other DNA
cttcaccacatggttgatgataa

SEQ ID NO: 224 - DNA, 23 bp, synthetic construct
  misc_feature 1..23: note = Description of Artificial Sequence: Syntheticprimer
  source 1..23: mol_type = other DNA
ctccaccacatggttgatgataa

SEQ ID NO: 225 - DNA, 23 bp, synthetic construct
  misc_feature 1..23: note = Description of Artificial Sequence: Syntheticprimer
  source 1..23: mol_type = other DNA
ctccaccacatggttgacgataa

SEQ ID NO: 226 - DNA, 23 bp, synthetic construct
  misc_feature 1..23: note = Description of Artificial Sequence: Syntheticprimer
  source 1..23: mol_type = other DNA
ctccaccacatggtagatgataa

SEQ ID NO: 227 - DNA, 23 bp, synthetic construct
  misc_feature 1..23: note = Description of Artificial Sequence: Syntheticprimer
  source 1..23: mol_type = other DNA
cttcaccacatggtagatgataa

SEQ ID NO: 228 - DNA, 21 bp, synthetic construct
  misc_feature 1..21: note = Description of Artificial Sequence: Syntheticprimer
  source 1..21: mol_type = other DNA
ttctggtacacctggttttgg

SEQ ID NO: 229 - DNA, 21 bp, synthetic construct
  misc_feature 1..21: note = Description of Artificial Sequence: Syntheticprimer
  source 1..21: mol_type = other DNA
ttctggcacacctggttttgg

SEQ ID NO: 230 - DNA, 21 bp, synthetic construct
  misc_feature 1..21: note = Description of Artificial Sequence: Syntheticprimer
  source 1..21: mol_type = other DNA
ttctggaacacctggttttgg

SEQ ID NO: 231 - DNA, 21 bp, synthetic construct
  misc_feature 1..21: note = Description of Artificial Sequence: Syntheticprimer
  source 1..21: mol_type = other DNA
ttctgggacacctggttttgg

SEQ ID NO: 232 - DNA, 21 bp, synthetic construct
  misc_feature 1..21: note = Description of Artificial Sequence: Syntheticprimer
  source 1..21: mol_type = other DNA
ttctggtacaccaggctttgg

SEQ ID NO: 233 - DNA, 21 bp, synthetic construct
  misc_feature 1..21: note = Description of Artificial Sequence: Syntheticprimer
  source 1..21: mol_type = other DNA
ttctggtacccctggttttgg

SEQ ID NO: 234 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 235 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 236 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 237 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 238 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 239 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 240 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 241 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 242 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 243 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 244 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 245 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 246 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 247 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 248 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 249 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 250 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 251 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 252 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 253 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 254 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 255 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 256 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 257 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 258 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 259 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 260 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 261 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 262 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 263 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 264 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 265 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 266 - DNA, 40 bp, synthetic construct; identical to SEQ ID NO: 23
  misc_feature 1..40: note = Description of Artificial Sequence: Syntheticprimer
  source 1..40: mol_type = other DNA

SEQ ID NO: 267 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 268 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 269 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 270 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 271 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 272 - DNA, 23 bp, synthetic construct
  misc_feature 1..23: note = Description of Artificial Sequence: Syntheticprimer
  source 1..23: mol_type = other DNA
cctctctatgggcagtcggtgat

SEQ ID NO: 273 - DNA, 24 bp, synthetic construct
  misc_feature 1..24: note = Description of Artificial Sequence: Syntheticprimer
  source 1..24: mol_type = other DNA
gaaactacgcgagaatttcagaag

SEQ ID NO: 274 - DNA, 24 bp, synthetic construct
  misc_feature 1..24: note = Description of Artificial Sequence: Syntheticprimer
  source 1..24: mol_type = other DNA
gaaattacgcgagaatttcagaag

SEQ ID NO: 275 - DNA, 24 bp, synthetic construct
  misc_feature 1..24: note = Description of Artificial Sequence: Syntheticprimer
  source 1..24: mol_type = other DNA
gaaattatgcgagaatttcagaag

SEQ ID NO: 276 - DNA, 24 bp, synthetic construct
  misc_feature 1..24: note = Description of Artificial Sequence: Syntheticprimer
  source 1..24: mol_type = other DNA
gaaactatgcgagaatttcagagg

SEQ ID NO: 277 - DNA, 22 bp, synthetic construct
  misc_feature 1..22: note = Description of Artificial Sequence: Syntheticprimer
  source 1..22: mol_type = other DNA
cgaagaggtgcagcataagtag

SEQ ID NO: 278 - DNA, 22 bp, synthetic construct
  misc_feature 1..22: note = Description of Artificial Sequence: Syntheticprimer
  source 1..22: mol_type = other DNA
cgtaatggtgccgcgtatgttg

SEQ ID NO: 279 - DNA, 22 bp, synthetic construct
  misc_feature 1..22: note = Description of Artificial Sequence: Syntheticprimer
  source 1..22: mol_type = other DNA
cgtagaggtgcagaatacgttg

SEQ ID NO: 280 - DNA, 24 bp, synthetic construct
  misc_feature 1..24: note = Description of Artificial Sequence: Syntheticprimer
  source 1..24: mol_type = other DNA
ccaatatggaagacatcgtaaacg

SEQ ID NO: 281 - DNA, 21 bp, synthetic construct
  misc_feature 1..21: note = Description of Artificial Sequence: Syntheticprimer
  source 1..21: mol_type = other DNA
ccaattccgtatcggtttatc

SEQ ID NO: 282 - DNA, 21 bp, synthetic construct
  misc_feature 1..21: note = Description of Artificial Sequence: Syntheticprimer
  source 1..21: mol_type = other DNA
ccaattccgtattggtttatc

SEQ ID NO: 283 - DNA, 20 bp, synthetic construct
  misc_feature 1..20: note = Description of Artificial Sequence: Syntheticprimer
  source 1..20: mol_type = other DNA
acttccatttgagcacgttc

SEQ ID NO: 284 - DNA, 20 bp, synthetic construct
  misc_feature 1..20: note = Description of Artificial Sequence: Syntheticprimer
  source 1..20: mol_type = other DNA
acttccatttgggcacgttc

SEQ ID NO: 285 - DNA, 20 bp, synthetic construct
  misc_feature 1..20: note = Description of Artificial Sequence: Syntheticprimer
  source 1..20: mol_type = other DNA
acttccatttgtgcacgttc

SEQ ID NO: 286 - DNA, 21 bp, synthetic construct
  misc_feature 1..21: note = Description of Artificial Sequence: Syntheticprimer
  source 1..21: mol_type = other DNA
gtgaacgtgctcaaatggaag

SEQ ID NO: 287 - DNA, 21 bp, synthetic construct
  misc_feature 1..21: note = Description of Artificial Sequence: Syntheticprimer
  source 1..21: mol_type = other DNA
gtgaacgtgcccaaatggaag

SEQ ID NO: 288 - DNA, 21 bp, synthetic construct
  misc_feature 1..21: note = Description of Artificial Sequence: Syntheticprimer
  source 1..21: mol_type = other DNA
gtgaacgtgcacaaatggaag

SEQ ID NO: 289 - DNA, 23 bp, synthetic construct
  misc_feature 1..23: note = Description of Artificial Sequence: Syntheticprimer
  source 1..23: mol_type = other DNA
acatagctatcttcttcatcagc

SEQ ID NO: 290 - DNA, 23 bp, synthetic construct
  misc_feature 1..23: note = Description of Artificial Sequence: Syntheticprimer
  source 1..23: mol_type = other DNA
acgtaactatcctcttcatcagc

SEQ ID NO: 291 - DNA, 23 bp, synthetic construct
  misc_feature 1..23: note = Description of Artificial Sequence: Syntheticprimer
  source 1..23: mol_type = other DNA
acatagctatcctcttcatcagc

SEQ ID NO: 292 - DNA, 23 bp, synthetic construct
  misc_feature 1..23: note = Description of Artificial Sequence: Syntheticprimer
  source 1..23: mol_type = other DNA
acatagctatcttcttcgtcagc

SEQ ID NO: 293 - DNA, 23 bp, synthetic construct
  misc_feature 1..23: note = Description of Artificial Sequence: Syntheticprimer
  source 1..23: mol_type = other DNA
acataactgtcttcttcatcagc

SEQ ID NO: 294 - DNA, 20 bp, synthetic construct
  misc_feature 1..20: note = Description of Artificial Sequence: Syntheticprimer
  source 1..20: mol_type = other DNA
tcggtgagatggaggtatgg

SEQ ID NO: 295 - DNA, 20 bp, synthetic construct
  misc_feature 1..20: note = Description of Artificial Sequence: Syntheticprimer
  source 1..20: mol_type = other DNA
tcggtgagatggaagtatgg

SEQ ID NO: 296 - DNA, 20 bp, synthetic construct
  misc_feature 1..20: note = Description of Artificial Sequence: Syntheticprimer
  source 1..20: mol_type = other DNA
tcggtgaaatggaagtatgg

SEQ ID NO: 297 - DNA, 21 bp, synthetic construct
  misc_feature 1..21: note = Description of Artificial Sequence: Syntheticprimer
  source 1..21: mol_type = other DNA
ctcggaatgattctggaacac

SEQ ID NO: 298 - DNA, 21 bp, synthetic construct
  misc_feature 1..21: note = Description of Artificial Sequence: Syntheticprimer
  source 1..21: mol_type = other DNA
ctcggaatgattcaggaacac

SEQ ID NO: 299 - DNA, 18 bp, synthetic construct
  misc_feature 1..18: note = Description of Artificial Sequence: Syntheticprimer
  source 1..18: mol_type = other DNA
gaagaagcagttcggcaa

SEQ ID NO: 300 - DNA, 17 bp, synthetic construct
  misc_feature 1..17: note = Description of Artificial Sequence: Syntheticprimer
  source 1..17: mol_type = other DNA
gagtcgatgacgatcat

SEQ ID NO: 301 - DNA, 18 bp, synthetic construct
  misc_feature 1..18: note = Description of Artificial Sequence: Syntheticprimer
  source 1..18: mol_type = other DNA
ccacgctcacgctgcagg

SEQ ID NO: 302 - DNA, 18 bp, synthetic construct
  misc_feature 1..18: note = Description of Artificial Sequence: Syntheticprimer
  source 1..18: mol_type = other DNA
cgagcccgagcgcaccag

SEQ ID NO: 303 - DNA, 17 bp, synthetic construct
  misc_feature 1..17: note = Description of Artificial Sequence: Syntheticprimer
  source 1..17: mol_type = other DNA
cgaaggcgagatgggcg

SEQ ID NO: 304 - DNA, 16 bp, synthetic construct
  misc_feature 1..16: note = Description of Artificial Sequence: Syntheticprimer
  source 1..16: mol_type = other DNA
tcgagacgcaccgacg

SEQ ID NO: 305 - DNA, 19 bp, synthetic construct
  misc_feature 1..19: note = Description of Artificial Sequence: Syntheticprimer
  source 1..19: mol_type = other DNA
gtgcaggcgaagatcgtcg

SEQ ID NO: 306 - DNA, 17 bp, synthetic construct
  misc_feature 1..17: note = Description of Artificial Sequence: Syntheticprimer
  source 1..17: mol_type = other DNA
ccatcgcctcggcttcg

SEQ ID NO: 307 - DNA, 21 bp, synthetic construct
  misc_feature 1..21: note = Description of Artificial Sequence: Syntheticprimer
  source 1..21: mol_type = other DNA
ggcggacgtcacactccattc

SEQ ID NO: 308 - DNA, 24 bp, synthetic construct
  misc_feature 1..24: note = Description of Artificial Sequence: Syntheticprimer
  source 1..24: mol_type = other DNA
ccgtcttcgatagcgattgggtgg

SEQ ID NO: 309 - DNA, 23 bp, synthetic construct
  misc_feature 1..23: note = Description of Artificial Sequence: Syntheticprimer
  source 1..23: mol_type = other DNA
ggttgctcgtgaagacatccaac

SEQ ID NO: 310 - DNA, 23 bp, synthetic construct
  misc_feature 1..23: note = Description of Artificial Sequence: Syntheticprimer
  source 1..23: mol_type = other DNA
caccagtaacgtctgttgtacgg

SEQ ID NO: 311 - DNA, 25 bp, synthetic construct
  misc_feature 1..25: note = Description of Artificial Sequence: Syntheticprimer
  source 1..25: mol_type = other DNA
caggcgatgatgttccagttatcgc

SEQ ID NO: 312 - DNA, 24 bp, synthetic construct
  misc_feature 1..24: note = Description of Artificial Sequence: Syntheticprimer
  source 1..24: mol_type = other DNA
gtagcaacagtaccacgtccagtg

SEQ ID NO: 313 - DNA, 38 bp, synthetic construct
  misc_feature 1..38: note = Description of Artificial Sequence: Syntheticprimer
  source 1..38: mol_type = other DNA
tgtaaaacgacggccagtgcnggrtcyttytcytgrca

SEQ ID NO: 314 - DNA, 18 bp, synthetic construct
  misc_feature 1..18: note = Description of Artificial Sequence: Syntheticprimer
  source 1..18: mol_type = other DNA
tgtaaaacgacggccagt

SEQ ID NO: 315 - DNA, 37 bp, synthetic construct
  misc_feature 1..37: note = Description of Artificial Sequence: Syntheticprimer
  source 1..37: mol_type = other DNA
caggaaacagctatgaccaygsnggnggnaarttyra

SEQ ID NO: 316 - DNA, 18 bp, synthetic construct
  misc_feature 1..18: note = Description of Artificial Sequence: Syntheticprimer
  source 1..18: mol_type = other DNA
caggaaacagctatgacc

SEQ ID NO: 317 - DNA, 19 bp, synthetic construct
  misc_feature 1..19: note = Description of Artificial Sequence: Syntheticprimer
  source 1..19: mol_type = other DNA
cagctgggacatcctggcc

SEQ ID NO: 318 - DNA, 24 bp, synthetic construct
  misc_feature 1..24: note = Description of Artificial Sequence: Syntheticprimer
  source 1..24: mol_type = other DNA
tgagggatgttgttggtaaagcac

SEQ ID NO: 319 - DNA, 24 bp, synthetic construct
  misc_feature 1..24: note = Description of Artificial Sequence: Syntheticprimer
  source 1..24: mol_type = other DNA
gtgctttaccaacaacatccctca

SEQ ID NO: 320 - DNA, 23 bp, synthetic construct
  misc_feature 1..23: note = Description of Artificial Sequence: Syntheticprimer
  source 1..23: mol_type = other DNA
tgtctttggtctgggagctgaac

SEQ ID NO: 321 - DNA, 60 bp, synthetic construct
  misc_feature 1..60: note = Description of Artificial Sequence: Syntheticprimer
  modified_base 48: mod_base = i
  source 1..60: mol_type = other DNA
ccatctcatccctgcgtgtctccgactcagacgctcgacaagyggcgnacgggtgagtaa

SEQ ID NO: 322 - DNA, 60 bp, synthetic construct
  misc_feature 1..60: note = Description of Artificial Sequence: Syntheticprimer
  modified_base 48: mod_base = i
  source 1..60: mol_type = other DNA
ccatctcatccctgcgtgtctccgactcagagacgcactcagyggcgnacgggtgagtaa

SEQ ID NO: 323 - DNA, 60 bp, synthetic construct
  misc_feature 1..60: note = Description of Artificial Sequence: Syntheticprimer
  modified_base 48: mod_base = i
  source 1..60: mol_type = other DNA
ccatctcatccctgcgtgtctccgactcagagcactgtagagyggcgnacgggtgagtaa

SEQ ID NO: 324 - DNA, 60 bp, synthetic construct
  misc_feature 1..60: note = Description of Artificial Sequence: Syntheticprimer
  modified_base 48: mod_base = i
  source 1..60: mol_type = other DNA
ccatctcatccctgcgtgtctccgactcagatcagacacgagyggcgnacgggtgagtaa

SEQ ID NO: 325 - DNA, 60 bp, synthetic construct
  misc_feature 1..60: note = Description of Artificial Sequence: Syntheticprimer
  modified_base 48: mod_base = i
  source 1..60: mol_type = other DNA
ccatctcatccctgcgtgtctccgactcagatatcgcgagagyggcgnacgggtgagtaa

SEQ ID NO: 326 - DNA, 60 bp, synthetic construct
  misc_feature 1..60: note = Description of Artificial Sequence: Syntheticprimer
  modified_base 48: mod_base = i
  source 1..60: mol_type = other DNA
ccatctcatccctgcgtgtctccgactcagcgtgtctctaagyggcgnacgggtgagtaa

SEQ ID NO: 327 - DNA, 60 bp, synthetic construct
  misc_feature 1..60: note = Description of Artificial Sequence: Syntheticprimer
  modified_base 48: mod_base = i
  source 1..60: mol_type = other DNA
ccatctcatccctgcgtgtctccgactcagctcgcgtgtcagyggcgnacgggtgagtaa

SEQ ID NO: 328 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagacgctcgacaactcctacgggaggcagcag

SEQ ID NO: 329 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagagacgcactcactcctacgggaggcagcag

SEQ ID NO: 330 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagagcactgtagactcctacgggaggcagcag

SEQ ID NO: 331 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagatcagacacgactcctacgggaggcagcag

SEQ ID NO: 332 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagatatcgcgagactcctacgggaggcagcag

SEQ ID NO: 333 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagcgtgtctctaactcctacgggaggcagcag

SEQ ID NO: 334 - DNA, 60 bp, synthetic construct
  misc_feature 1..60: note = Description of Artificial Sequence: Syntheticprimer
  source 1..60: mol_type = other DNA
ccatctcatccctgcgtgtctccgactcagacgagtgcgtactcctacgggaggcagcag

SEQ ID NO: 335 - DNA, 47 bp, synthetic construct
  misc_feature 1..47: note = Description of Artificial Sequence: Syntheticprimer
  source 1..47: mol_type = other DNA
acactgacgacatggttctacaaaactcggtcatttagaggaagtaa

SEQ ID NO: 336 - DNA, 41 bp, synthetic construct
  misc_feature 1..41: note = Description of Artificial Sequence: Syntheticprimer
  source 1..41: mol_type = other DNA
tacggtagcagagacttggtctgctgcgttcttcatcgatg

SEQ ID NO: 337 - DNA, 42 bp, synthetic construct
  misc_feature 1..42: note = Description of Artificial Sequence: Syntheticprimer
  source 1..42: mol_type = other DNA
acactgacgacatggttctacaatcgagtctttgaacgcaca

SEQ ID NO: 338 - DNA, 42 bp, synthetic construct
  misc_feature 1..42: note = Description of Artificial Sequence: Syntheticprimer
  source 1..42: mol_type = other DNA
tacggtagcagagacttggtctcctacctgatccgaggtcaa